(19) United States
(12) Patent Application Publication
(10) Pub. No.: US 2025/0152781 A1
(43) Pub. Date: May 15, 2025

(54) TISSUE ENGINEERING AND DRUG DELIVERY DEVICE

(71) Applicant: Ohio State Innovation Foundation, Columbus, OH (US)

(72) Inventors: Francisco CHAPARRO, Phoenix, AZ (US); John LANNUTTI; Marco COUTINHO DA SILVA, Worthington, OH (US)

(21) Appl. No.: 18/727,300

(22) PCT Filed: Jan. 9, 2023
(86) PCT No.: PCT/US2023/060328

§ 371 Date: Jul. 8, 2024

Related U.S. Application Data
(60) Provisional application No. 63/297,435, filed on Jan. 7, 2022.

(51) Int. Cl. A61L 27/38; A61L 27/34

(52) U.S. Cl. CPC A61L 27/38; A61L 27/34; A61L 2420/00

(57) ABSTRACT

Described herein are compositions and devices for the delivery of active agents.

### CROSS-REFERENCE TO RELATED APPLICATIONS

The application claims the benefit of U.S. Provisional Application No. 63/297,435, filed Jan. 7, 2022, which is hereby incorporated herein by reference in its entirety.

### BACKGROUND

Tissue engineering scaffolds have long been used to provide mechanical and chemical support to mammalian cells for purposes of organ regeneration. However, their ability to deliver drugs and or cells within their interior to a site of clinical interest has been limited by the fact that the same space that allows ‘packaging’ of these drugs or cells compromises their mechanical properties.

There is a need for a scaffolds that can be used as drug delivery devices as well as for tissue engineering.

The compositions and methods disclosed herein address these and other needs.

### SUMMARY

Provided herein are drug delivery devices comprising: a matrix comprising a wall and a lumen extending therethrough from a first end to a second end, and an active agent disposed within the lumen of the matrix. The matrix can be formed from a biodegradable polymer and optionally a non-biodegradable polymer. The matrix can have a porosity of at least 30%, as determined by mercury porosimetry or apparent density. The matrix can have a density of at least 0.25 g/c, as determined by mercury porosimetry or apparent density. In some embodiments, at least a portion of the device can include a coating including a population of cells. In some embodiments, the cells are selected to improve biocompatibility or drug delivery with the surrounding tissue. In some embodiments, the cells are stem cells, endothelial cells, mesenchymal cells, kidney cells, pancreatic cells, liver cells, or specific cancer cells engineered to produce desirable compounds or microvesicles.

In some embodiments, the biodegradable polymer can include polycaprolactone (PCL). In some embodiments, the non-biodegradable polymer can include polyethylene terephthalate (PET).

In some embodiments, the matrix can have a wall thickness of from 25 microns to 1500 microns. In some embodiments, the lumen can have a diameter of from 100 μm to 6.0 mm. In some embodiments, the matrix can have a length of 0.1 cm to 20 cm. In some embodiments, the wall can further include a plurality of pores. In some embodiments, the pores can have an average diameter of from 100 nm to 10 μm.

In some embodiments, the drug delivery device can release the active agent over a period of at least 3 days, at least 30 days, at least 3 months, at least 6 months when incubated in phosphate buffered saline at 37° C.

In some embodiments, the active agent can be present in an amount of from 1 μg/ml to 100,000 μg/ml in the drug delivery device.

Disclosed herein are also methods for preparing a drug delivery device including a matrix including a wall and a lumen extending therethrough from a first end to a second end, and an active agent disposed within the lumen of the matrix, the method comprising: forming a matrix by forming a wall on a rod, the wall including a biodegradable polymer and optionally a non-biodegradable polymer; sintering the matrix following forming the wall; and injecting an active agent dispersed in a carrier into the lumen of the matrix. In some embodiments, forming the wall can include electrospinning using a solution of the biodegradable polymer and optionally a non-biodegradable polymer. In some embodiments, forming the wall can include electrospinning using a solution of the biodegradable polymer and optionally a non-biodegradable polymer, and a voltage difference of from 10 kV to 30 kV. In some embodiments, the solution can further include a porogen.

In some embodiments, when the solution includes the biodegradable polymer and the non-biodegradable polymer, the biodegradable polymer can be present in a concentration of from 5% to 95%, in the solution. In some embodiments, when the solution includes the biodegradable polymer and the non-biodegradable polymer, the non-biodegradable polymer can be present in a concentration of from 5% to 95% in the solution.

In some embodiments, sintering can include heating at a temperature of from 50° C. to 150° C. for a period of from 1 minute to 6 hours. In some embodiments, the method can further include washing the matrix following sintering. In some embodiments, the method can further include drying the matrix following washing. In some embodiments, drying can be in vacuo at a temperature of from 50° C. to 150° C. for a period of from 1 minute to 6 hours. In some embodiments, injecting is performed after sintering. In some embodiments, the injecting step is performed after drying.

In some embodiments, the method can further include culturing a population of cells from a subject on the wall of the matrix prior to injection of the active agent into the lumen of the matrix. In some embodiments, the drug delivery device described herein are made by the methods described herein.

Described herein are also methods of forming a graft for implantation into a subject, the method can include culturing a population of cells from the subject on the wall of the matrix to form a graft for implantation into the subject. In some embodiments, culturing is performed in a bioreactor. In some embodiments, the cells are stem cells, endothelial cells, mesenchymal cells, kidney cells, pancreatic cells, liver cells, or specific cancer cells engineered to produce desirable compounds or microvesicles.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 depicts a hollow tube created via electrospinning onto a solid metal mandrel.

FIG. 2 depicts a hollow plastic tube formed by sintering a hollow tube of electrospun fiber to full or nearly-full density.

FIG. 3A-3C depicts (3A) Heat sealed dense tubing original consisting of electrospun fiber. (3B) A fully sealed tube containing a mixture of silicone oil and Rose Bengal. (3C) Purpose-built heat sealer (TTS-8 Tube Heat Sealer, U Heat Seal Solutions, Corona, CA) used to seal these tubes.

FIG. 4 depicts release rate of Rose Bengal through a PCL capsule wall formed by densifying electrospun PCL using sintering.

FIG. 5 depicts diffusive release of Rose Bengal through a 95% PCL-5% polyethylene terephthalate (PET) wall formed by electrospinning followed by sintering to full density. Drug release rates above 10 μg/day are observed even after 30+ days of release.

FIG. 6 depicts an exemplary drug delivery device 100 including a tubular matrix 101 with two closed ends 102a and 102b including a wall 103 and a lumen 104 extending therethrough from a first end to a second end, and an active agent disposed within the lumen 104 of the tubular matrix 101.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms and do not exclude additional elements or steps. Although the terms “comprising” and “including” have been used herein to describe various embodiments, the terms “consisting essentially of” and “‘consisting of’ can be used in place of “comprising” and “including” to provide for more specific embodiments of the invention and are also disclosed. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of“one or more,” “at least one,” and “one or more than one.” Therefore, an element preceded by “a” or “an” does not, without more constraints, preclude the existence of additional identical elements. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraocular, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a natural or synthetically derived chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, a symptom of a disease or disorder, or preventing or altering a physiological process. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial agent or agents can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced or increased as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n— COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include companion or domesticated animals (e.g., cats, dogs, horses etc.), livestock (e.g., cattle, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### Drug Delivery Device

The drug delivery device described herein can include a matrix with two closed ends including a wall and a lumen extending therethrough, and an active agent disposed within the lumen of the matrix. In some embodiments, the matrix can have any suitable geometry including, but not limited to, tubular, cylindrical, hexagonal, square, square tubular, hexagonal tubular, cylindrical tubular. In some embodiments, the matrix can be tubular.

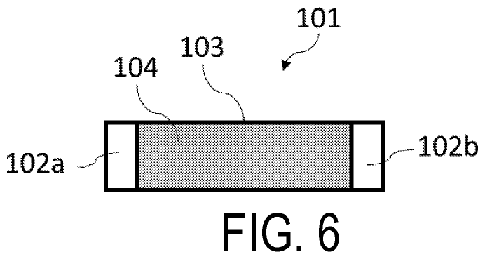

An exemplary the drug delivery device 100 is shown in FIG. 6. The drug delivery device 100 can include a tubular matrix 101 with two closed ends 102a and 102b including a wall 103 and a lumen 104 extending therethrough from a first end to a second end, and an active agent disposed within the lumen 104 of the tubular matrix 101. In some embodiments, the wall 103 of the tubular matrix 101 can be formed from a biodegradable polymer. In some embodiments, the wall 103 of the tubular matrix 101 can further include a non-biodegradable polymer.

The matrix can be formed from a biodegradable polymer. In some embodiments, the biodegradable polymer can include a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof. In some embodiments, the biodegradable polymer can include polycaprolactone (PCL).

In some embodiments, the wall can further include a non-biodegradable polymer. In some embodiments, the non-biodegradable polymer can include polyethylene terephthalate (PET), polyvinyl chloride (PVC), polyethylene (PE), polysulfone (PSU), polyethersulfone (PES), polypropylene (PP), polystyrene (PS), poly(urethanes), poly(acrylates), poly(ethylene vinyl acetate), nylon, copolymers, or blends thereof. In some embodiments, the non-biodegradable polymer can include polyethylene terephthalate (PET).

In some embodiments, at least a portion of the device can include a coating including a population of cells. In some embodiments, the cells are stem cells, endothelial cells, mesenchymal cells, kidney cells, pancreatic cells, liver cells, or specific cancer cells engineered to produce desirable compounds or microvesicles.

The matrix can have a porosity of at least 5% as determined by mercury porosimetry or apparent density (e.g., at least 10%, at least 20%, at least 30%, at least 40%, at least 50%, or at least 60%). The matrix can have a porosity of 70% or less as determined by mercury porosimetry or apparent density (e.g., 60% or less, 50% or less, 40% or less, 30% or less, 20% or less, or 10% or less).

The matrix can have a porosity ranging from any of the minimum values described above to any of the maximum values described above. For example, in some embodiments, the matrix can have a porosity of from 5% to 70% as determined by mercury porosimetry or apparent density (e.g., from 5% to 60%, from 5% to 50%, from 5% to 40%, from 5% to 30%, from 5% to 20%, from 5% to 10%, from 10% to 70%, from 10% to 60%, from 10% to 50%, from 10% to 40% from 10% 30%, from 10% to 20%, from 20% to 30%, from 20% to 40%, from 20% to 50%, from 20% to 60%, from 20% to 70%, from 30% to 70%, from 30% to 60%, from 30% to 50%, from 30% to 40%, from 40% to 70%, from 40% to 60% from 40% to 50%, from 50% to 60%, from 50% 70%, or from 60% to 70%).

The matrix can have a density of at least 0.25 g/c as determined by mercury porosimetry or apparent density (e.g., at least 0.35 g/c, at least 0.45 g/c, or at least 0.65 g/c). The matrix can have a density of 0.70 g/c or less as determined by mercury porosimetry or apparent density (e.g., 0.65 g/c or less, 0.60 g/c or less, 0.55 g/c or less, 0.50 g/c or less, 0.45 g/c or less, 0.40 g/c or less, 0.35 g/c or less, or 0.30 g/c or less).

The matrix can have a density ranging from any of the minimum values described above to any of the maximum values described above. For example, in some embodiments, the matrix can have a density of from 0.25 g/c to 0.70 g/c as determined by mercury porosimetry or apparent density, (e.g., from 0.25 g/c to 0.60 g/c, from 0.25 g/c to 0.50 g/c, from 0.25 g/c to 0.40 g/c, from 0.25 g/c to 0.30 g/c, from 0.3 g/c to 0.60 g/c, from 0.3 g/c to 0.50 g/c, from 0.3 g/c to 0.40 g/c, from 0.35 g/c to 0.60 g/c, from 0.35 g/c to 0.50 g/c, from 0.35 g/c to 0.40 g/c, from 0.4 g/c to 0.60 g/c, from 0.4 g/c to 0.50 g/c, from 0.50 g/c to 0.60 g/c, from 0.50 g/c to 0.70 g/c, from 0.40 g/c to 0.70 g/c, or from 0.30 g/c to 0.70 g/c).

The matrix can have a porosity of at least 5% (e.g., at least 10%, at least 20%, at least 30%, at least 40%, at least 50%, or at least 60%) and a density of at least 0.25 g/c (e.g., at least 0.35 g/c, at least 0.45 g/c, or at least 0.65 g/c) as determined by mercury porosimetry or apparent density.

The matrix can have a porosity of 70% or less (e.g., 60% or less, 50% or less, 40% or less, 30% or less, 20% or less, or 10% or less) and a density of 0.70 g/c or less (e.g., 0.65 g/c or less, 0.60 g/c or less, 0.55 g/c or less, 0.50 g/c or less, 0.45 g/c or less, 0.40 g/c or less, 0.35 g/c or less, or 0.30 g/c or less) as determined by mercury porosimetry or apparent density.

The matrix can have a porosity and a density ranging from any of the minimum values described above to any of the maximum values described above. For example, in some embodiments, the matrix can have a porosity of from 5% to 70% (e.g., from 5% to 60%, from 5% to 50%, from 5% to 40%, from 5% to 30%, from 5% to 20%, from 5% to 10%, from 10% to 70%, from 10% to 60%, from 10% to 50%, from 10% to 40% from 10% 30%, from 10% to 20%, from 20% to 30%, from 20% to 40%, from 20% to 50%, from 20% to 60%, from 20% to 70%, from 30% to 70%, from 30% to 60%, from 30% to 50%, from 30% to 40%, from 40% to 70%, from 40% to 60% from 40% to 50%, from 50% to 60%, from 50% 70%, or from 60% to 70%) and a density of from 0.25 g/c to 0.70 g/c, (e.g., from 0.25 g/c to 0.60 g/c, from 0.25 g/c to 0.50 g/c, from 0.25 g/c to 0.40 g/c, from 0.25 g/c to 0.30 g/c, from 0.3 g/c to 0.60 g/c, from 0.3 g/c to 0.50 g/c, from 0.3 g/c to 0.40 g/c, from 0.35 g/c to 0.60 g/c, from 0.35 g/c to 0.50 g/c, from 0.35 g/c to 0.40 g/c, from 0.4 g/c to 0.60 g/c, from 0.4 g/c to 0.50 g/c, from 0.50 g/c to 0.60 g/c, from 0.50 g/c to 0.70 g/c, from 0.40 g/c to 0.70 g/c, or from 0.30 g/c to 0.70 g/c) as determined by mercury porosimetry or apparent density.

The matrix can have a wall thickness of at least 25 microns, (e.g., at least 50 microns, at least 100 microns, at least 250 microns, at least 500 microns, at least 750 microns, at least 1000 microns, or at least 1250 microns). The matrix can have a wall thickness of 1500 microns or less, (e.g., 1250 microns or less, 1000 microns or less, 750 microns or less, 500 microns or less, 250 microns or less, 100 microns or less, or 50 microns or less).

The matrix can have a wall thickness ranging from any of the minimum values described above to any of the maximum values described above. For example, in some embodiments, the matrix can have a wall thickness of from 25 microns to 1500 microns, such as from 25 microns to 100 microns, from 25 microns to 250 microns, from 25 microns to 500 microns, from 25 microns to 750 microns, from 25 microns to 1000 microns, from 25 microns to 1250 microns, from 25 microns to 1500 microns, from 50 microns to 100 microns, from 50 microns to 250 microns, from 50 microns to 500 microns, from 50 microns to 750 microns, from 50 microns to 1000 microns, from 50 microns to 1250 microns, from 50 microns to 1500 microns, from 100 microns to 500 microns, from 100 microns to 750 microns, from 100 microns to 1000 microns, from 100 microns to 1250 microns, from 100 microns to 1500 microns, from 250 microns to 500 microns, from 250 microns to 750 microns, from 250 microns to 1000 microns, from 250 microns to 1250 microns, from 250 microns to 1500 microns, from 500 microns to 750 microns, microns, from 500 microns to 1000 microns, from 500 microns to 1250 microns, from 500 microns to 1500 microns, or from 1000 microns to 1500 microns.

The lumen can have a diameter of at least 100 μm, (e.g., at least 250 μm, at least 500 μm, at least 1 nm, at least 10 nm, at least 25 nm, at least 50 nm, at least 75 nm, at least 100 nm, at least 500 nm, at least 1 mm, at least 2 mm, at least 3 mm, at least 4 mm, or at least 5 mm). The lumen can have a diameter of 6 mm or less, (e.g., 5 mm or less, 4 mm or less, 3 mm or less, 2 mm or less, 1 mm or less, 500 nm or less, 250 nm or less, 100 nm or less, 50 nm or less, 25 nm or less, 10 nm or less, 1 nm or less, 500 μm or less, or 250 μm or less)

The matrix can have a wall thickness ranging from any of the minimum values described above to any of the maximum values described above. For example, in some embodiments, the lumen can have a diameter of from 100 μm to 6.0 mm, such as from 100 μm to 1 nm, from 100 μm to 10 nm, from 100 μm to 25 nm, from 100 μm to 50 nm, from 100 μm to 100 nm, from 100 μm to 500 nm, from 100 μm to 1.0 mm, from 100 μm to 2.0 mm, from 100 μm to 4.0 mm, from 100 μm to 5 mm, from 500 μm to 1 nm, from 500 μm to 10 nm, from 500 μm to 25 nm, from 500 μm to 50 nm, from 500 μm to 100 nm, from 500 μm to 500 nm, from 500 μm to 1 mm, from 500 μm to 2 mm, from 500 μm to 4 mm, from 500 μm to 5 mm, from 500 μm to 6 mm, from 1 nm to 10 nm, from 1 nm to 25 nm, from 1 nm to 50 nm, from 1 nm to 100 nm, from 1 nm to 500 nm, from 1 nm to 1 mm, from 1 nm to 2 mm, from 1 nm to 4 mm, from 1 nm to 5 mm, from 1 nm to 6 mm, from 25 nm to 50 nm, from 25 nm to 100 nm, from 25 nm to 500 nm, from 25 nm to 1 mm, from 25 nm to 2 mm, from 25 nm to 4 mm, from 25 nm to 5 mm, from 25 nm to 6 mm, from 50 nm to 100 nm, from 50 nm to 500 nm, from 50 nm to 1 mm, from 50 nm to 2 mm, from 50 nm to 4 mm, from 50 nm to 5 mm, from 50 nm to 6 mm, from 100 nm to 500 nm, from 100 nm to 1 mm, from 100 nm to 2 mm, from 100 nm to 4 mm, from 100 nm to 5 mm, from 100 nm to 6 mm, from 500 nm to 1 mm, from 500 nm to 2 mm, from 500 nm to 4 mm, from 500 nm to 5 mm, from 500 nm to 6 mm, from 1 mm to 2 mm, from 1 mm to 3 mm, from 1 nm to 4 mm, from 1 mm to 5 mm, from 1 mm to 6 mm, from 2 mm to 3 mm, from 2 nm to 4 mm, from 2 mm to 5 mm, from 2 mm to 6 mm, from 3 nm to 4 mm, from 3 mm to 5 mm, from 3 mm to 6 mm, from 4 mm to 5 mm, from 4 mm to 6 mm, or from 5 mm to 6 mm.

The matrix can have a length of at least 0.1 cm, (e.g., at least 0.5 cm, at least 1 cm, at least 2 cm, at least 3 cm, at least 4 cm, at least 5 cm, at least 6 cm, at least 7 cm, at least 8 cm, at least 9 cm, at least 10 cm, at least 11 cm, at least 12 cm, at least 13 cm, at least 14 cm, at least 15 cm, at least 16 cm, at least 17 cm, at least 18 cm, or at least 19 cm). The matrix can have a length of 20 cm or less, (e.g., 19 cm or less, 18 cm or less, 17 cm or less, 16 cm or less, 15 cm or less, 14 cm or less, 13 cm or less, 12 cm or less, 11 cm or less, 10 cm or less, 9 cm or less, 8 cm or less, 7 cm or less, 6 cm or less, 5 cm or less, 4 cm or less, 3 cm or less, 2 cm or less, 1 cm or less, or 0.5 cm or less).

The matrix can have a length ranging from any of the minimum values described above to any of the maximum values described above. For example, in some embodiments, the matrix can have a length of 0.1 cm to 20 cm, such as from 0.5 cm to 20 cm, from 1 cm to 20 cm, from 5 cm to 20 cm, from 10 cm to 20 cm, from 15 cm to 20 cm, from 0.5 cm to 15 cm, from 1 cm to 15 cm, from 5 cm to 15 cm, from 10 cm to 15 cm, from 0.5 cm to 10 cm, from 1 cm to 10 cm, from 5 cm to 10 cm, from 0.5 cm to 2 cm, from 0.5 cm to 5 cm, from 1 cm to 2 cm, from 1 cm to 3 cm, from 1 cm to 5 cm, or from 0.5 cm to 1 cm.

In some embodiments, the wall can further include a plurality of pores. The pores can have an average diameter of at least 100 nm, (e.g., at least 0.5 nm, at least 1 μm, or at least 5 μm). The pores can have an average diameter of 10 μm or less (e.g., 5 μm or less, 1 μm or less, or 0.5 μm or less).

The pores can have an average diameter ranging from any of the minimum values described above to any of the maximum values described above. For example, in some embodiments, the pores can have an average diameter of from 100 nm to 10 μm, such as from 100 nm to 500 nm, from 100 nm to 1 μm, from 100 nm to 5 μm from 500 nm to 1 μm, from 500 nm to 5 μm, from 500 nm to 10 μm, from 1 μm to 5 μm, from 1 μm to 10 μm, or from 5 μm to 10 μm.

In some embodiments, the drug delivery device can be in the form of a capsule.

The capsule can have a length of at least 0.1 cm, (e.g., at least 0.5 cm, at least 1 cm, at least 2 cm, at least 3 cm, or at least 4 cm). The capsule can have a length of 5 cm or less, (e.g., 4 cm or less, 3 cm or less, 2 cm or less, 1 cm or less, or 0.5 cm or less).

The capsule can have a length ranging from any of the minimum values described above to any of the maximum values described above. For example, in some embodiments, the capsule can have a length of from 0.1 cm to 5 cm, such as from 0.1 cm to 4 cm, from 0.1 cm to 3 cm, from 0.1 cm to 2 cm, from 0.1 cm to 1 cm, from 0.1 cm to 0.5 cm, from 0.5 cm to 5 cm, from 0.5 cm to 4 cm, from 0.5 cm to 3 cm, from 0.5 cm to 2 cm, from 0.5 cm to 1 cm, from 1 cm to 5 cm, from 1 cm to 4 cm, from 1 cm to 3 cm, from 1 cm to 2 cm, from 2 cm to 4 cm, from 2 cm to 3 cm, from 3 cm to 4 cm, from 3 cm to 5 cm, from 2 cm to 5 cm, or from 4 cm to 5 cm.

The capsule can have an inner diameter of at least 100 μm, (e.g., at least 250 μm, at least 500 μm, at least 750 μm, at least 1000 μm, at least 1500 μm, or at least 1750 μm). The capsule can have an inner diameter of 2000 μm or less, (e.g., 1750 μm or less, 1500 μm or less, 1250 μm or less, 1000 μm or less, 750 μm or less, 500 μm or less, or 250 μm or less).

The capsule can have an inner diameter ranging from any of the minimum values described above to any of the maximum values described above. For example, in some embodiments, the capsule can have an inner diameter of from 100 μm to 2000 μm, such as from 100 μm to 1500 μm, from 100 μm to 1000 μm, from 100 μm to 750 μm, from 100 μm to 500 μm, from 100 μm to 250 μm, from 250 μm to 2000 μm, from 250 μm to 1500 μm, from 250 μm to 1000 μm, from 250 μm to 750 μm, from 250 μm to 500 μm, from 500 μm to 2000 μm, from 500 μm to 1500 μm, from 500 μm to 1000 μm, from 500 μm to 750 μm, from 1000 μm to 2000 μm, from 1000 μm to 1500 μm, from 1500 μm to 1750 μm, from 1500 μm to 2000 μm, from 200 μm to 1000 μm, or from 200 μm to 800 μm.

The capsule can have an outer diameter of at least 50 μm greater than the inner diameter, (e.g., at least 100 μm, at least 150 μm, at least 200 μm, or at least 250 μm). The capsule can have an outer diameter of 300 μm or less greater than the inner diameter, (e.g., 250 μm or less, 200 μm or less, 150 μm or less, or 100 μm or less).

The capsule can have an outer diameter ranging from any of the minimum values described above to any of the maximum values described above. For example, in some embodiments, the capsule can have an outer diameter of from 50 μm to 300 μm greater than the inner diameter, such as from 50 μm to 100 μm, from 50 μm to 200 μm, from 100 μm to 200 μm, from 100 μm to 300 μm, or from 200 μm to 300 μm.

The drug delivery device releases the active agent over a period of at least 3 days, (e.g., at least 1 week, at least 2 weeks, at least 3 weeks, at least 30 days, at least 3 months, or at least 6 months) when incubated in phosphate-buffered saline at 37° C. The drug delivery device releases the active agent over a period of 6 months or less, (e.g., 3 months or less, 30 days or less, 3 weeks or less, 2 weeks or less, or 1 week or less) when incubated in phosphate-buffered saline at 37° C.

The drug delivery device releases the active agent over a period ranging from any of the minimum values described above to any of the maximum values described above. For example, in some embodiments, the drug delivery device releases the active agent over a period of from 3 days to 12 months, (e.g., from 3 days to 9 months, from 3 days to 6 months, from 3 days to 3 months, from 3 days to 30 days, from 3 days to 2 weeks, from 1 week to 2 weeks, from 1 week to 3 weeks, from 1 week to 30 days, from 1 week to 3 months, from 1 week to 6 months, from 1 week to 9 months, from 1 week to 12 months, from 2 weeks to 3 weeks, from 2 weeks to 30 days, from 2 weeks to 3 months, from 2 weeks to 6 months, from 2 weeks to 9 months, from 2 weeks to 12 months, from 3 weeks to 30 days, from 3 weeks to 3 months, from 3 weeks to 6 months, from 3 weeks to 9 months, from 3 weeks to 12 months, from 30 days to 3 months, from 30 days to 6 months, from 30 days to 9 months, 30 days to 12 months, from 3 months to 6 months, from 3 months to 9 months, from 3 months to 12 months, from 6 months to 9 months, from 6 months to 12 months, from 9 months to 12 months) when incubated in phosphate-buffered saline at 37° C.

### Active Agents

In some embodiments, the drug delivery device can include an active agent disposed within the lumen of the matrix. In some embodiments, the active agent can be present in amount of from 1 μg/ml to 100,000 μg/ml (e.g., 1 μg/ml to 50,000 μg/ml, 1 μg/ml to 10,000 μg/ml, 1 μg/ml to 1,000 μg/ml, 1 μg/ml to 250 μg/ml, 1 μg/ml to 50 μg/ml, 1 μg/ml to 10 μg/ml, 1 μg/ml to 5 μg/ml, 10 μg/ml to 50,000 μg/ml, 10 μg/ml to 10,000 μg/ml, 10 μg/ml to 1,000 μg/ml, 10 μg/ml to 250 μg/ml, 10 μg/ml to 50 μg/ml, 50 μg/ml to 50,000 μg/ml, 50 μg/ml to 10,000 μg/ml, 50 μg/ml to 1,000 μg/ml, 50 μg/ml to 250 μg/ml, 250 μg/ml to 50,000 μg/ml, 250 μg/ml to 10,000 μg/ml, 250 μg/ml to 1,000 μg/ml, 1000 μg/ml to 50,000 μg/ml, 1000 μg/ml to 10,000 μg/ml, or 10000 μg/ml to 50,000 μg/ml).

As used herein, a “active agent” or “therapeutic agents” refers to one or more therapeutic agents, active ingredients, or substances that can be used to treat a medical condition of the eye, a cancer, heart, reproduction, or contraception. As discussed herein, the therapeutic agents can be released from the disclosed compositions in a biologically active form.

It is further understood, that as used herein, the terms “therapeutic agent” includes any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

In some embodiments, the therapeutic agent may include an agent useful as contraceptive such as natural and synthetic progestins, estrogens, or any combination thereof. For example, suitable progestins include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. For example suitable estrogens include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators.

In some embodiments, the therapeutic agent may include gonodotropin releasing hormone (GnRh) agonist, anologs, antagonist, bioconjugate or pharmaceutically acceptable salts or prodrugs, thereof such as deslorelin, histrelin, avorelin, leuprolide, triptorelin, nafarelin, goserelin, buserelin, and fertirelin; or an immunocontraceptive agent based on zona pellucida (ZP) (i.e., porcine zona pellucida) or gonadotropin-releasing hormone (GnRH); or GnRH-based bioconjugates.

In some embodiments, the therapeutic agent may include an agent useful in the treatment of an ophthalmological disorder or an eye disease such as: beta-blockers including timolol, betaxolol, levobetaxolol, and carteolol; miotics including pilocarpine; carbonic anhydrase inhibitors; serotonergics; muscarinics; dopaminergic agonists; adrenergic agonists including apraclonidine and brimonidine; anti-angiogenesis agents; anti-infective agents including quinolones such as ciprofloxacin and aminoglycosides such as tobramycin and gentamicin; non-steroidal and steroidal anti-inflammatory agents, such as suprofen, diclofenac, ketorolac, rimexolone and tetrahydrocortisol; growth factors, such as EGF; immunosuppressant agents; and anti-allergic agents including olopatadine; prostaglandins such as latanoprost; 15-keto latanoprost; travoprost; and unoprostone isopropyl.

In some embodiments, the therapeutic agent is selected from the group consisting of an anti-inflammatory agent, a calcineurin inhibitor, an antibiotic, a nicotinic acetylcholine receptor agonist, and an anti-lymphangiogenic agent. In some embodiments, the anti-inflammatory agent may be cyclosporine. In some embodiments, the calcineurin inhibitor may be voclosporin. In some embodiments, the antibiotic may be selected from the group consisting of amikacin, gentamycin, kanamycin, neomycin, netilmicin, streptomycin, tobramycin, teicoplanin, vancomycin, azithromycin, clarithromycin, dirithromycin, erythromycin, roxithromycin, troleandomycin, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, mafenide, sulfacetamide, sulfamethizole, sulfasalazine, sulfisoxazole, trimethoprim, cotrimoxazole, demeclocycline, doxycycline, minocycline, oxytetracycline, and tetracycline. In some embodiments, the nicotinic acetylcholine receptor agonist may be any of pilocarpine, atropine, nicotine, epibatidine, lobeline, or imidacloprid. In some embodiments, the anti-lymphangiogenic agent may be a vascular endothelial growth factor C (VEGF-C) antibody, a VEGF-D antibody or a VEGF-3 antibody.

In some aspects, the therapeutic agent may be selected from: a beta-blocker, including levobunolol (BETAGAN), timolol (BETIMOL, TIMOPTIC), betaxolol (BETOPTIC) and metipranolol (OPTIPRANOLOL); alpha-agonists, such as apraclonidine (IOPIDINE) and brimonidine (ALPHAGAN); carbonic anhydrase inhibitors, such as acetazolamide, methazolamide, dorzolamide (TRUSOPT) and brinzolamide (AZOPT); prostaglandins or prostaglandin analogs such as latanoprost (XALATAN), bimatoprost (LUMIGAN) and travoprost (TRAVATAN); miotic or cholinergic agents, such as pilocarpine (ISOPTO CARPINE, PILOPINE) and carbachol (ISOPTO CARBACHOL); epinephrine compounds, such as dipivefrin (PROPINE); forskolin; or neuroprotective compounds, such as brimonidine and memantine; a steroid derivative, such as 2-methoxyestradiol or analogs or derivatives thereof; or an antibiotic.

The term “VEGF” refers to a vascular endothelial growth factor that induces angiogenesis or an angiogenic process, including, but not limited to, increased permeability. As used herein, the term “VEGF” includes the various subtypes of VEGF (also known as vascular permeability factor (VPF) and VEGF-A) that arise by, e.g., alternative splicing of the VEGF-ANPF gene including VEGF121, VEGF165 and VEGF189. Further, as used herein, the term “VEGF” includes VEGF-related angiogenic factors such as PIGF (placental growth factor), VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1), VEGFR-2 (KDR/Flk-1), or VEGFR-3 (FLT-4). The term “VEGF” can be used to refer to a “VEGF” polypeptide or a “VEGF” encoding gene or nucleic acid.

The term “anti-VEGF agent” refers to an agent that reduces, or inhibits, either partially or fully, the activity or production of a VEGF. An anti-VEGF agent can directly or indirectly reduce or inhibit the activity or production of a specific VEGF such as VEGF165. Furthermore, “anti-VEGF agents” include agents that act on either a VEGF ligand or its cognate receptor so as to reduce or inhibit a VEGF-associated receptor signal. Non-limiting examples of “anti-VEGF agents” include antisense molecules, ribozymes or RNAi that target a VEGF nucleic acid; anti-VEGF aptamers, anti-VEGF antibodies to VEGF itself or its receptor, or soluble VEGF receptor decoys that prevent binding of a VEGF to its cognate receptor; antisense molecules, ribozymes, or RNAi that target a cognate VEGF receptor (VEGFR) nucleic acid; anti-VEGFR aptamers or anti-VEGFR antibodies that bind to a cognate VEGFR receptor; and VEGFR tyrosine kinase inhibitors.

In some embodiments, the therapeutic agent may include an anti-VEGF agent. Representative examples of anti-VEGF agents include ranibizumab, bevacizumab, aflibercept, KH902 VEGF receptor-Fc, fusion protein, 2C3 antibody, ORA102, pegaptanib, bevasiranib, SIRNA-027, decursin, decursinol, picropodophyllin, guggulsterone, PLG101, eicosanoid LXA4, PTK787, pazopanib, axitinib, CDDO-Me, CDDO-Imm, shikonin, beta-, hydroxyisovalerylshikonin, ganglioside GM3, DC101 antibody, Mab25 antibody, Mab73 antibody, 4A5 antibody, 4E10 antibody, 5F12 antibody, VA01 antibody, BL2 antibody, VEGF-related protein, sFLT01, sFLTO2, Peptide B3, TG100801, sorafenib, G6-31 antibody, a fusion antibody and an antibody that binds to an epitope of VEGF. Additional non-limiting examples of anti-VEGF agents useful in the present methods include a substance that specifically binds to one or more of a human vascular endothelial growth factor-A (VEGF-A), human vascular endothelial growth factor-B (VEGF-B), human vascular endothelial growth factor-C(VEGF-C), human vascular endothelial growth factor-D (VEGF-D) and human vascular endothelial growth, factor-E (VEGF-E), and an antibody that binds, to an epitope of VEGF.

In various aspects, the anti-VEGF agent is the antibody ranibizumab or a pharmaceutically acceptable salt thereof. Ranibizumab is commercially available under the trademark LUCENTIS. In another embodiment, the anti-VEGF agent is the antibody bevacizumab or a pharmaceutically acceptable salt thereof. Bevacizumab is commercially available under the trademark AVASTIN. In another embodiment, the anti-VEGF agent is aflibercept or a pharmaceutically acceptable salt thereof. Aflibercept is commercially available under the trademark EYLEA. In one embodiment, the anti-VEGF agent is pegaptanib or a pharmaceutically acceptable salt thereof. Pegaptinib is commercially available under the trademark MACUGEN. In another embodiment, the anti-VEGF agent is an antibody or an antibody fragment that binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of the three-dimensional structure of VEGF that is displayed, such that the epitope is exposed on the surface of the folded VEGF molecule. In one embodiment, the epitope is a linear amino acid sequence from VEGF.

In various aspects, the therapeutic agent may include an agent that blocks or inhibits VEGF-mediated activity, e.g., one or more VEGF antisense nucleic acids. The present disclosure provides the therapeutic or prophylactic use of nucleic acids comprising at least six nucleotides that are antisense to a gene or cDNA encoding VEGF or a portion thereof. As used herein, a VEGF “antisense” nucleic acid refers to a nucleic acid capable of hybridizing by virtue of some sequence complementarity to a portion of an RNA (preferably mRNA) encoding VEGF. The antisense nucleic acid may be complementary to a coding and/or noncoding region of an mRNA encoding VEGF. Such antisense nucleic acids have utility as compounds that prevent VEGF expression and can be used in the treatment of diabetes. The antisense nucleic acids of the disclosure are double-stranded or single-stranded oligonucleotides, RNA or DNA or a modification or derivative thereof, and can be directly administered to a cell or produced intracellularly by transcription of exogenous, introduced sequences.

The VEGF antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. In addition, the antisense molecules may be polymers that are nucleic acid mimics, such as PNA, morpholino oligos, and LNA. Other types of antisense molecules include short double-stranded RNAs, known as siRNAs, and short hairpin RNAs, and long dsRNA (>50 bp but usually ≥500 bp).

In various aspects, the therapeutic agent may include one or more ribozyme molecule designed to catalytically cleave gene mRNA transcripts encoding VEGF, preventing translation of target gene mRNA and, therefore, expression of the gene product.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage event. The composition of ribozyme molecules must include one or more sequences complementary to the target gene mRNA and must include the well-known catalytic sequence responsible for mRNA cleavage. For this sequence, see, e.g., U.S. Pat. No. 5,093,246. While ribozymes that cleave mRNA at site-specific recognition sequences can be used to destroy mRNAs encoding VEGF, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art. The ribozymes of the present disclosure also include RNA endoribonucleases (hereinafter “Cech-type ribozymes”) such as the one that occurs naturally in Tetrahymena thermophila (known as the IVS, or L-19 IVS RNA). The Cech-type ribozymes have an eight base pair active site that hybridizes to a target RNA sequence where after cleavage of the target RNA takes place. The disclosure encompasses those Cech-type ribozymes that target eight base-pair active site sequences that are present in the gene encoding VEGF.

In further aspects, the therapeutic agent may include an antibody that inhibits VEGF such as bevacizumab or ranibizumab. In still further aspects, therapeutic agent may include an agent that inhibits VEGF activity such as a tyrosine kinases stimulated by VEGF, examples of which include, but are not limited to lapatinib, sunitinib, sorafenib, axitinib, and pazopanib.

The term “anti-RAS agent” or “anti-Renin Angiotensin System agent” refers to refers to an agent that reduces, or inhibits, either partially or fully, the activity or production of a molecule of the renin angiotensin system (RAS). Non-limiting examples of “anti-RAS” or “anti-Renin Angiotensin System” molecules are one or more of an angiotensin-converting enzyme (ACE) inhibitor, an angiotensin-receptor blocker, and a renin inhibitor.

In some embodiments, the therapeutic agent may include a renin angiotensin system (RAS) inhibitor. In some embodiments, the renin angiotensin system (RAS) inhibitor is one or more of an angiotensin-converting enzyme (ACE) inhibitor, an angiotensin-receptor blocker, and a renin inhibitor.

Non limiting examples of angiotensin-converting enzyme (ACE) inhibitors which are useful in the present invention include, but are not limited to: alacepril, alatriopril, altiopril calcium, ancovenin, benazepril, benazepril hydrochloride, benazeprilat, benzazepril, benzoylcaptopril, captopril, captoprilcysteine, captoprilglutathione, ceranapril, ceranopril, ceronapril, cilazapril, cilazaprilat, converstatin, delapril, delaprildiacid, enalapril, enalaprilat, enalkiren, enapril, epicaptopril, foroxymithine, fosfenopril, fosenopril, fosenopril sodium, fosinopril, fosinopril sodium, fosinoprilat, fosinoprilic acid, glycopril, hemorphin-4, idapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spirapril hydrochloride, temocapril, temocapril hydrochloride, teprotide, trandolapril, trandolaprilat, utibapril, zabicipril, zabiciprilat, zofenopril, zofenoprilat, pharmaceutically acceptable salts thereof, and mixtures thereof.

Non limiting examples of angiotensin-receptor blockers which are useful in the present invention include, but are not limited to: irbesartan (U.S. Pat. No. 5,270,317, hereby incorporated by reference in its entirety), candesartan (U.S. Pat. Nos. 5,196,444 and 5,705,517 hereby incorporated by reference in their entirety), valsartan (U.S. Pat. No. 5,399,578, hereby incorporated by reference in its entirety), and losartan (U.S. Pat. No. 5,138,069, hereby incorporated by reference in its entirety).

Non limiting examples of renin inhibitors which may be used as therapeutic agents include, but are not limited to: aliskiren, ditekiren, enalkiren, remikiren, terlakiren, ciprokiren and zankiren, pharmaceutically acceptable salts thereof, and mixtures thereof.

The term “steroid” refers to compounds belonging to or related to the following illustrative families of compounds: corticosteroids, mineralicosteroids, and sex steroids (including, for example, potentially androgenic or estrogenic or anti-androgenic and anti-estrogenic molecules). Included among these are, for example, prednisone, prednisolone, methyl-prednisolone, triamcinolone, fluocinolone, aldosterone, spironolactone, danazol (otherwise known as OPTINA), and others. In some embodiments, the therapeutic agent may include a steroid.

The terms “peroxisome proliferator-activated receptor gamma agent,” or “PPAR-γ agent,” or “PPARG agent,” or “PPAR-gamma agent” refers to agents which directly or indirectly act upon the peroxisome proliferator-activated receptor. This agent may also influence PPAR-alpha, “PPARA” activity.

In some embodiments, the therapeutic agent may include a modulator of macrophage polarization. Illustrative modulators of macrophage polarization include peroxisome proliferator activated receptor gamma (PPAR-g) modulators, including, for example, agonists, partial agonists, antagonists or combined PPAR-gamma/alpha agonists. In some embodiments, the therapeutic agent may include a PPAR gamma modulator, including PPAR gamma modulators that are full agonists or a partial agonists. In some embodiments, the PPAR gamma modulator is a member of the drug class of thiazolidinediones (TZDs, or glitazones). By way of non-limiting example, the PPAR gamma modulator may be one or more of rosiglitazone (AVANDIA), pioglitazone (ACTOS), troglitazone (REZULIN), netoglitazone, rivoglitazone, ciglitazone, rhodanine. In some embodiments, the PPAR gamma modulator is one or more of irbesartan and telmesartan. In some embodiments, the PPAR gamma modulator is a nonsteroidal anti-inflammatory drug (NSAID, such as, for example, ibuprofen) or an indole. Known inhibitors include the experimental agent GW-9662. Further examples of PPAR gamma modulators are described in WIPO Publication Nos. WO/1999/063983, WO/2001/000579, Nat Rev Immunol. 2011 Oct. 25; 11(11):750-61, or agents identified using the methods of WO/2002/068386, the contents of which are hereby incorporated by reference in their entireties.

In some embodiments, the PPAR gamma modulator is a “dual,” or “balanced,” or “pan” PPAR modulator. In some embodiments, the PPAR gamma modulator is a glitazar, which bind two or more PPAR isoforms, e.g., muraglitazar (Pargluva) and tesaglitazar (Galida) and aleglitazar.

In some embodiments, the therapeutic agent may include semapimod (CNI-1493) as described in Bianchi, et al. (March 1995). Molecular Medicine (Cambridge, Mass.) 1 (3): 254-266, the contents of which is hereby incorporated by reference in its entirety.

In some embodiments, the therapeutic agent may include a migration inhibitory factor (MIF) inhibitor. Illustrative MIF inhibitors are described in WIPO Publication Nos. WO 2003/104203, WO 2007/070961, WO 2009/117706 and U.S. Pat. Nos. 7,732,146 and 7,632,505, and 7,294,753 7,294,753 the contents of which are hereby incorporated by reference in their entireties. In some embodiments, the MIF inhibitor is (S,R)-3-(4-hydroxyphenyl)-4,5-dihydro-5-isoxazole acetic acid methyl ester (ISO-1), isoxazoline, p425 (J. Biol. Chem., 287, 30653-30663), epoxyazadiradione, or vitamin E.

In some embodiments, the therapeutic agent may include a chemokine receptor 2 (CCR2) inhibitor as described in, for example, U.S. patent and Patent Publication Nos.: U.S. Pat. Nos. 7,799,824, 8,067,415, US 2007/0197590, US 2006/0069123, US 2006/0058289, and US 2007/0037794, the contents of which are hereby incorporated by reference in their entireties. In some embodiments, the CCR2) inhibitor is Maraviroc, cenicriviroc, CD192, CCX872, CCX140, 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide, vicriviroc, SCH351125, TAK779, Teijin, RS-504393, compound 2, compound 14, or compound 19 (Plos ONE 7(3): e32864).

In some embodiments, the therapeutic agent may include an agent that modulates autophagy, microautophagy, mitophagy or other forms of autophagy. In some embodiments, the therapeutic agent may include sirolimus, tacrolimis, rapamycin, everolimus, bafilomycin, chloroquine, hydroxychloroquine, spautin-1, metformin, perifosine, resveratrol, trichostatin, valproic acid, Z-VAD-FMK, or others known to those in the art. Without wishing to be bound by theory, agent that modulates autophagy, microautophagy, mitophagy or other forms of autophagy may alter the recycling of intra-cellular components, for example, but not limited to, cellular organelles, mitochondria, endoplasmic reticulum, lipids or others. Without further wishing to be bound by theory, this agent may or may not act through microtubule-associated protein 1A/1B-light chain 3 (LC3).

In some embodiments, the therapeutic agent may include an agent used to treat cancer, i.e., a cancer drug or anti-cancer agent. Exemplary cancer drugs can be selected from antimetabolite anti-cancer agents and antimitotic anti-cancer agents, and combinations thereof, to a subject. Various antimetabolite and antimitotic anti-cancer agents, including single such agents or combinations of such agents, may be employed in the methods and compositions described herein.

Antimetabolic anti-cancer agents typically structurally resemble natural metabolites, which are involved in normal metabolic processes of cancer cells such as the synthesis of nucleic acids and proteins. The antimetabolites, however, differ enough from the natural metabolites such that they interfere with the metabolic processes of cancer cells. In the cell, antimetabolites are mistaken for the metabolites they resemble, and are processed by the cell in a manner analogous to the normal compounds. The presence of the “decoy” metabolites prevents the cells from carrying out vital functions and the cells are unable to grow and survive. For example, antimetabolites may exert cytotoxic activity by substituting these fraudulent nucleotides into cellular DNA, thereby disrupting cellular division, or by inhibition of critical cellular enzymes, which prevents replication of DNA.

In one aspect, therefore, the antimetabolite anti-cancer agent is a nucleotide or a nucleotide analog. In certain aspects, for example, the antimetabolite agent may include purine (e.g., guanine or adenosine) or analogs thereof, or pyrimidine (cytidine or thymidine) or analogs thereof, with or without an attached sugar moiety.

Suitable antimetabolite anti-cancer agents for use in the present disclosure may be generally classified according to the metabolic process they affect, and can include, but are not limited to, analogues and derivatives of folic acid, pyrimidines, purines, and cytidine. Thus, in one aspect, the antimetabolite agent(s) is selected from the group consisting of cytidine analogs, folic acid analogs, purine analogs, pyrimidine analogs, and combinations thereof.

In one particular aspect, for example, the antimetabolite agent is a cytidine analog. According to this aspect, for example, the cytidine analog may be selected from the group consisting of cytarabine (cytosine arabinodside), azacitidine (5-azacytidine), and salts, analogs, and derivatives thereof.

In another particular aspect, for example, the antimetabolite agent is a folic acid analog. Folic acid analogs or antifolates generally function by inhibiting dihydrofolate reductase (DHFR), an enzyme involved in the formation of nucleotides: when this enzyme is blocked, nucleotides are not formed, disrupting DNA replication and cell division. According to certain aspects, for example, the folic acid analog may be selected from the group consisting of denopterin, methotrexate (amethopterin), pemetrexed, pteropterin, raltitrexed, trimetrexate, and salts, analogs, and derivatives thereof.

In another particular aspect, for example, the antimetabolite agent is a purine analog. Purine-based antimetabolite agents function by inhibiting DNA synthesis, for example, by interfering with the production of purine containing nucleotides, adenine and guanine which halts DNA synthesis and thereby cell division. Purine analogs can also be incorporated into the DNA molecule itself during DNA synthesis, which can interfere with cell division. According to certain aspects, for example, the purine analog may be selected from the group consisting of acyclovir, allopurinol, 2-aminoadenosine, arabinosyl adenine (ara-A), azacitidine, azathiprine, 8-aza-adenosine, 8-fluoro-adenosine, 8-methoxy-adenosine, 8-oxo-adenosine, cladribine, deoxycoformycin, fludarabine, gancylovir, 8-aza-guanosine, 8-fluoro-guanosine, 8-methoxy-guanosine, 8-oxo-guanosine, guanosine diphosphate, guanosine diphosphate-beta-L-2-aminofucose, guanosine diphosphate-D-arabinose, guanosine diphosphate-2-fluorofucose, guanosine diphosphate fucose, mercaptopurine (6-MP), pentostatin, thiamiprine, thioguanine (6-TG), and salts, analogs, and derivatives thereof.

In yet another particular aspect, for example, the antimetabolite agent is a pyrimidine analog. Similar to the purine analogs discussed above, pyrimidine-based antimetabolite agents block the synthesis of pyrimidine-containing nucleotides (cytosine and thymine in DNA; cytosine and uracil in RNA). By acting as “decoys,” the pyrimidine-based compounds can prevent the production of nucleotides, and/or can be incorporated into a growing DNA chain and lead to its termination. According to certain aspects, for example, the pyrimidine analog may be selected from the group consisting of ancitabine, azacitidine, 6-azauridine, bromouracil (e.g., 5-bromouracil), capecitabine, carmofur, chlorouracil (e.g. 5-chlorouracil), cytarabine (cytosine arabinoside), cytosine, dideoxyuridine, 3′-azido-3′-deoxythymidine, 3′-dideoxycytidin-2′-ene, 3′-deoxy-3′-deoxythymidin-2′-ene, dihydrouracil, doxifluridine, enocitabine, floxuridine, 5-fluorocytosine, 2-fluorodeoxycytidine, 3-fluoro-3′-deoxythymidine, fluorouracil (e.g., 5-fluorouracil (also known as 5-FU), gemcitabine, 5-methylcytosine, 5-propynylcytosine, 5-propynylthymine, 5-propynyluracil, thymine, uracil, uridine, and salts, analogs, and derivatives thereof. In one aspect, the pyrimidine analog is other than 5-fluorouracil. In another aspect, the pyrimidine analog is gemcitabine or a salt thereof.

In certain aspects, the antimetabolite agent is selected from the group consisting of 5-fluorouracil, capecitabine, 6-mercaptopurine, methotrexate, gemcitabine, cytarabine, fludarabine, pemetrexed, and salts, analogs, derivatives, and combinations thereof. In other aspects, the antimetabolite agent is selected from the group consisting of capecitabine, 6-mercaptopurine, methotrexate, gemcitabine, cytarabine, fludarabine, pemetrexed, and salts, analogs, derivatives, and combinations thereof. In one particular aspect, the antimetabolite agent is other than 5-fluorouracil. In a particularly preferred aspect, the antimetabolite agent is gemcitabine or a salt or thereof (e.g., gemcitabine HCl (Gemzar®)).

Other antimetabolite anti-cancer agents may be selected from, but are not limited to, the group consisting of acanthifolic acid, aminothiadiazole, brequinar sodium, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, Wellcome EHNA, Merck & Co. EX-015, fazarabine, fludarabine phosphate, N-(2′-furanidyl)-5-fluorouracil, Daiichi Seiyaku FO-152, 5-FU-fibrinogen, isopropyl pyrrolizine, Lilly LY-188011; Lilly LY-264618, methobenzaprim, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, tiazofurin, Erbamont TIF, tyrosine kinase inhibitors, Taiho UFT and uricytin, among others.

In one aspect, the antimitotic agent is a microtubule inhibitor or a microtubule stabilizer. In general, microtubule stabilizers, such as taxanes and epothilones, bind to the interior surface of the beta-microtubule chain and enhance microtubule assembly by promoting the nucleation and elongation phases of the polymerization reaction and by reducing the critical tubulin subunit concentration required for microtubules to assemble. Unlike mictrotubule inhibitors, such as the vinca alkaloids, which prevent microtubule assembly, the microtubule stabilizers, such as taxanes, decrease the lag time and dramatically shift the dynamic equilibrium between tubulin dimers and microtubule polymers towards polymerization. In one aspect, therefore, the microtubule stabilizer is a taxane or an epothilone. In another aspect, the microtubule inhibitor is a vinca alkaloid.

In some embodiments, the therapeutic agent may include a taxane or derivative or analog thereof. The taxane may be a naturally derived compound or a related form, or may be a chemically synthesized compound or a derivative thereof, with antineoplastic properties. The taxanes are a family of terpenes, including, but not limited to paclitaxel (Taxol®) and docetaxel (Taxotere®), which are derived primarily from the Pacific yew tree, Taxus brevifolia, and which have activity against certain tumors, particularly breast and ovarian tumors. In one aspect, the taxane is docetaxel or paclitaxel. Paclitaxel is a preferred taxane and is considered an antimitotic agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

Also included are a variety of known taxane derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; deoxygenated paclitaxel compounds such as those described in U.S. Pat. No. 5,440,056; and taxol derivatives described in U.S. Pat. No. 5,415,869. As noted above, it further includes prodrugs of paclitaxel including, but not limited to, those described in WO 98/58927; WO 98/13059; and U.S. Pat. No. 5,824,701. The taxane may also be a taxane conjugate such as, for example, paclitaxel-PEG, paclitaxel-dextran, paclitaxel-xylose, docetaxel-PEG, docetaxel-dextran, docetaxel-xylose, and the like. Other derivatives are mentioned in “Synthesis and Anticancer Activity of Taxol Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elsevier, Amsterdam 1986), among other references. Each of these references is hereby incorporated by reference herein in its entirety.

Various taxanes may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267) (each of which is hereby incorporated by reference herein in its entirety), or obtained from a variety of commercial sources, including for example, Sigma-Aldrich Co., St. Louis, Mo.

Alternatively, the antimitotic agent can be a microtubule inhibitor; in one preferred aspect, the microtubule inhibitor is a vinca alkaloid. In general, the vinca alkaloids are mitotic spindle poisons. The vinca alkaloid agents act during mitosis when chromosomes are split and begin to migrate along the tubules of the mitosis spindle towards one of its poles, prior to cell separation. Under the action of these spindle poisons, the spindle becomes disorganized by the dispersion of chromosomes during mitosis, affecting cellular reproduction. According to certain aspects, for example, the vinca alkaloid is selected from the group consisting of vinblastine, vincristine, vindesine, vinorelbine, and salts, analogs, and derivatives thereof.

The antimitotic agent can also be an epothilone. In general, members of the epothilone class of compounds stabilize microtubule function according to mechanisms similar to those of the taxanes. Epothilones can also cause cell cycle arrest at the G2-M transition phase, leading to cytotoxicity and eventually apoptosis. Suitable epithiolones include epothilone A, epothilone B, epothilone C, epothilone D, epothilone E, and epothilone F, and salts, analogs, and derivatives thereof. One particular epothilone analog is an epothilone B analog, ixabepilone (Ixempra™).

In certain aspects, the antimitotic anti-cancer agent is selected from the group consisting of taxanes, epothilones, vinca alkaloids, and salts and combinations thereof. Thus, for example, in one aspect the antimitotic agent is a taxane. More preferably in this aspect the antimitotic agent is paclitaxel or docetaxel, still more preferably paclitaxel. In another aspect, the antimitotic agent is an epothilone (e.g., an epothilone B analog). In another aspect, the antimitotic agent is a vinca alkaloid.

Examples of cancer drugs that may be used in the present disclosure include, but are not limited to: thalidomide; platinum coordination complexes such as cisplatin (cis-DDP), oxaliplatin and carboplatin; anthracenediones such as mitoxantrone; substituted ureas such as hydroxyurea; methylhydrazine derivatives such as procarbazine (N-methylhydrazine, MIH); adrenocortical suppressants such as mitotane (o,p′-DDD) and aminoglutethimide; RXR agonists such as bexarotene; and tyrosine kinase inhibitors such as sunitimib and imatinib. Examples of additional cancer drugs include alkylating agents, antimetabolites, natural products, hormones and antagonists, and miscellaneous agents. Alternate names are indicated in parentheses. Examples of alkylating agents include nitrogen mustards such as mechlorethamine, cyclophosphainide, ifosfamide, melphalan sarcolysin) and chlorambucil; ethylenimines and methylmelamines such as hexamethylmelamine and thiotepa; alkyl sulfonates such as busulfan; nitrosoureas such as carmustine (BCNU), semustine (methyl-CCNU), lomustine (CCNU) and streptozocin (streptozotocin); DNA synthesis antagonists such as estramustine phosphate; and triazines such as dacarbazine (DTIC, dimethyl-triazenoimidazolecarboxamide) and temozolomide. Examples of antimetabolites include folic acid analogs such as methotrexate (amethopterin); pyrimidine analogs such as fluorouracin (5-fluorouracil, 5-FU, SFU), floxuridine (fluorodeoxyuridine, FUdR), cytarabine (cytosine arabinoside) and gemcitabine; purine analogs such as mercaptopurine (6-mercaptopurine, 6-MP), thioguanine (6-thioguanine, TG) and pentostatin (2′-deoxycoformycin, deoxycoformycin), cladribine and fludarabine; and topoisomerase inhibitors such as amsacrine. Examples of natural products include vinca alkaloids such as vinblastine (VLB) and vincristine; taxanes such as paclitaxel, protein bound paclitaxel (Abraxane) and docetaxel (Taxotere); epipodophyllotoxins such as etoposide and teniposide; camptothecins such as topotecan and irinotecan; antibiotics such as dactinomycin (actinomycin D), daunorubicin (daunomycin, rubidomycin), doxorubicin, histrelin, bleomycin, mitomycin (mitomycin C), idarubicin, epiubicin; enzymes such as L-asparaginase; and biological response modifiers such as interferon alpha and interlelukin 2. Examples of hormones and antagonists include luteinising releasing hormone agonists such as buserelin; adrenocorticosteroids such as prednisone and related preparations; progestins such as hydroxyprogesterone caproate, medroxyprogesterone acetate and megestrol acetate; estrogens such as diethylstilbestrol and ethinyl estradiol and related preparations; estrogen antagonists such as tamoxifen and anastrozole; androgens such as testosterone propionate and fluoxymesterone and related preparations; androgen antagonists such as flutamide and bicalutamide; and gonadotropin-releasing hormone analogs such as leuprolide. Alternate names and trade-names of these and additional examples of cancer drugs, and their methods of use including dosing and administration regimens, will be known to a person versed in the art.

In some aspects, the anti-cancer agent may include a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these aspects, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate aspect of the invention.

According to certain aspects, the therapeutic agent may include a biologic drug, particularly an antibody. According to some aspects, the antibody is selected from the group consisting of cetuximab, anti-CD24 antibody, panitumumab and bevacizumab.

Therapeutic agents as used in the present disclosure may include peptides, proteins such as hormones, enzymes, antibodies, monoclonal antibodies, antibody fragments, monoclonal antibody fragments, and the like, nucleic acids such as aptamers, siRNA, DNA, RNA, antisense nucleic acids or the like, antisense nucleic acid analogs or the like, low-molecular weight compounds, or high-molecular-weight compounds, receptor agonists, receptor antagonists, partial receptor agonists, and partial receptor antagonists.

Additional representative therapeutic agents may include, but are not limited to, peptide drugs, protein drugs, desensitizing materials, antigens, factors, growth factors, anti-infective agents such as antibiotics, antimicrobial agents, antiviral, antibacterial, antiparasitic, antifungal substances and combination thereof, antiallergenics, steroids, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, cardioactive agents, nonsteroidal anti-inflammatory agents, antiparkinsonian agents, anti-Alzheimer's agents, antihypertensive agents, beta-adrenergic blocking agents, alpha-adrenergic blocking agents, nutritional agents, and the benzophenanthridine alkaloids. The therapeutic agent can further be a substance capable of acting as a stimulant, a sedative, a hypnotic, an analgesic, an anticonvulsant, and the like.

Additional therapeutic agents may include CNS-active drugs, neuro-active drugs, inflammatory and anti-inflammatory drugs, renal and cardiovascular drugs, gastrointestinal drugs, anti-neoplastics, immunomodulators, immunosuppressants, hematopoietic agents, growth factors, anticoagulant, thrombolytic, antiplatelet agents, hormones, hormone-active agents, hormone antagonists, vitamins, ophthalmic agents, anabolic agents, antacids, anti-asthmatic agents, anti-cholesterolemic and anti-lipid agents, anti-convulsants, anti-diarrheals, anti-emetics, anti-manic agents, antimetabolite agents, anti-nauseants, anti-obesity agents, anti-pyretic and analgesic agents, anti-spasmodic agents, anti-thrombotic agents, anti-tussive agents, anti-uricemic agents, anti-anginal agents, antihistamines, appetite suppressants, biologicals, cerebral dilators, coronary dilators, bronchiodilators, cytotoxic agents, decongestants, diuretics, diagnostic agents, erythropoietic agents, expectorants, gastrointestinal sedatives, hyperglycemic agents, hypnotics, hypoglycemic agents, laxatives, mineral supplements, mucolytic agents, neuromuscular drugs, peripheral vasodilators, psychotropics, stimulants, thyroid and anti-thyroid agents, tissue growth agents, uterine relaxants, vitamins, antigenic materials, and so on. Other classes of therapeutic agents include those cited in Goodman & Gilman's The Pharmacological Basis of Therapeutics (McGraw Hill) as well as therapeutic agents included in the Merck Index and The Physicians' Desk Reference (Thompson Healthcare).

Other therapeutic agents include androgen inhibitors, polysaccharides, growth factors (e.g., a vascular endothelial growth factor-VEGF), hormones, anti-angiogenesis factors, dextromethorphan, dextromethorphan hydrobromide, noscapine, carbetapentane citrate, chlophedianol hydrochloride, chlorpheniramine maleate, phenindamine tartrate, pyrilamine maleate, doxylamine succinate, phenyltoloxamine citrate, phenylephrine hydrochloride, phenylpropanolamine hydrochloride, pseudoephedrine hydrochloride, ephedrine, codeine phosphate, codeine sulfate morphine, mineral supplements, cholestryramine, N-acetylprocainamide, acetaminophen, aspirin, ibuprofen, phenyl propanolamine hydrochloride, caffeine, guaifenesin, aluminum hydroxide, magnesium hydroxide, peptides, polypeptides, proteins, amino acids, hormones, interferons, cytokines, and vaccines.

Further examples of therapeutic agents include, but are not limited to, peptide drugs, protein drugs, desensitizing materials, antigens, anti-infective agents such as antibiotics, antimicrobial agents, antiviral, antibacterial, antiparasitic, antifungal substances and combination thereof, antiallergenics, androgenic steroids, decongestants, hypnotics, steroidal anti-inflammatory agents, anti-cholinergics, sympathomimetics, sedatives, miotics, psychic energizers, tranquilizers, vaccines, estrogens, progestational agents, humoral agents, prostaglandins, analgesics, antispasmodics, antimalarials, antihistamines, antiproliferatives, anti-VEGF agents, cardioactive agents, nonsteroidal anti-inflammatory agents, antiparkinsonian agents, antihypertensive agents, β-adrenergic blocking agents, nutritional agents, and the benzophenanthridine alkaloids. The agent can further be a substance capable of acting as a stimulant, sedative, hypnotic, analgesic, anticonvulsant, and the like.

Further representative therapeutic agents include but are not limited to analgesics such as acetaminophen, acetylsalicylic acid, and the like; anesthetics such as lidocaine, xylocaine, and the like: anorexics such as dexadrine, phendimetrazine tartrate, and the like; antiarthritics such as methylprednisolone, ibuprofen, and the like; antiasthmatics such as terbutaline sulfate, theophylline, ephedrine, and the like; antibiotics such as sulfisoxazole, penicillin G, ampicillin, cephalosporins, amikacin, gentamicin, tetracyclines, chloramphenicol, erythromycin, clindamycin, isoniazid, rifampin, and the like; antifungals such as amphotericin B, nystatin, ketoconazole, and the like; antivirals such as acyclovir, amantadine, and the like; anticancer agents such as cyclophosphamide, methotrexate, etretinate, paclitaxel, taxol, and the like; anticoagulants such as heparin, warfarin, and the like; anticonvulsants such as phenyloin sodium, diazepam, and the like; antidepressants such as isocarboxazid, amoxapine, and the like; antihistamines such as diphenhydramine HCl, chlorpheniramine maleate, and the like; hormones such as insulin, progestins, estrogens, corticoids, glucocorticoids, androgens, and the like, tranquilizers such as thorazine, diazepam, chlorpromazine HCl, reserpine, chlordiazepoxide HCl, and the like; antispasmodics such as belladonna alkaloids, dicyclomine hydrochloride, and the like; vitamins and minerals such as essential amino acids, calcium, iron, potassium, zinc, vitamin B12, and the like; cardiovascular agents such as prazosin HCl, nitroglycerin, propranolol HCl, hydralazine HCl, pancrelipase, succinic acid dehydrogenase, and the like, peptides and proteins such as LHRH, somatostatin, calcitonin, growth hormone, glucagon-like peptides, growth releasing factor, angiotensin, FSH, EGF, bone morphogenic protein (BMP), erythopoeitin (EPO), interferon, interleukin, collagen, fibrinogen, insulin, Factor VIII, Factor IX, Enbrel®, Rituxam@, Herceptin®, alpha-glucosidase, Cerazyme/Ceredose®, vasopressin, ACTH, human serum albumin, gamma globulin, structural proteins, blood product proteins, complex proteins, enzymes, antibodies, monoclonal antibodies, and the like; prostaglandins; nucleic acids; carbohydrates; fats; narcotics such as morphine, codeine, and the like, psychotherapeutics; anti-malarials, L-dopa, diuretics such as furosemide, spironolactone, and the like; antiulcer drugs such as rantidine HCl, cimetidine HCl, and the like.

The therapeutic agent can also be an immunomodulator, including, for example, cytokines, interleukins, interferon, colony stimulating factor, tumor necrosis factor, and the like; immunosuppressants such as rapamycin, tacrolimus, and the like; allergens such as cat dander, birch pollen, house dust mite, grass pollen, and the like; antigens of bacterial organisms such as Streptococcus pneumoniae, Haemophilus influenzae, Staphylococcus aureus, Streptococcus pyrogenes, Corynebacterium diphteriae, Listeria monocytogenes, Bacillus anthracis, Clostridium tetani, Clostridium botulinum, Clostridium perfringens. Neisseria meningitides, Neisseria gonorrhoeae, Streptococcus mutans. Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptspirosis interrogans, Borrelia burgddorferi, Campylobacter jejuni, and the like; antigens of such viruses as smallpox, influenza A and B, respiratory synctial, parainfluenza, measles, HIV, SARS, varicella-zoster, herpes simplex 1 and 2, cytomeglavirus, Epstein-Barr, rotavirus, rhinovirus, adenovirus, papillomavirus, poliovirus, mumps, rabies, rubella, coxsackieviruses, equine encephalitis, Japanese encephalitis, yellow fever, Rift Valley fever, lymphocytic choriomeningitis, hepatitis B, and the like, antigens of such fungal, protozoan, and parasitic organisms such as Cryptococcus neoformans, Histoplasma capsulatum, Candida albicans, Candida tropicalis, Nocardia asteroids, Rickettsia ricketsii, Rickettsia typhi, Mycoplasma pneumoniae, Chlamydia psittaci, Chlamydia trachomatis, Plasmodium falciparum, Trypanasoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof.

In a further specific aspect, the therapeutic agent can include an antibiotic. The antibiotic can be, for example, one or more of Amikacin, Gentamicin, Kanamycin, Neomycin, Netilmicin, Streptomycin, Tobramycin, Paromomycin, Ansamycins, Geldanamycin, Herbimycin, Carbacephem, Loracarbef, Carbapenems, Ertapenem, Doripenem, Imipenem/Cilastatin, Meropenem, Cephalosporins (First generation), Cefadroxil, Cefazolin, Cefalotin or Cefalothin, Cefalexin, Cephalosporins (Second generation), Cefaclor, Cefamandole, Cefoxitin, Cefprozil, Cefuroxime, Cephalosporins (Third generation), Cefixime, Cefdinir, Cefditoren, Cefoperazone, Cefotaxime, Cefpodoxime, Ceftazidime, Ceftibuten, Ceftizoxime, Ceftriaxone, Cephalosporins (Fourth generation), Cefepime, Cephalosporins (Fifth generation), Ceftobiprole, Glycopeptides, Teicoplanin, Vancomycin, Macrolides, Azithromycin, Clarithromycin, Dirithromycin, Erythromycin, Roxithromycin, Troleandomycin, Telithromycin, Spectinomycin, Monobactams, Aztreonam, Penicillins, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Polypeptides, Bacitracin, Colistin, Polymyxin B, Quinolones, Ciprofloxacin, Enoxacin, Gatifloxacin, Levofloxacin, Lomefloxacin, Moxifloxacin, Norfloxacin, Ofloxacin, Trovafloxacin, Sulfonamides, Mafenide, Prontosil (archaic), Sulfacetamide, Sulfamethizole, Sulfanilimide (archaic), Sulfasalazine, Sulfisoxazole, Trimethoprim, Trimethoprim-Sulfamethoxazole (Co-trimoxazole) (TMP-SMX), Tetracyclines, including Demeclocycline, Doxycycline, Minocycline, Oxytetracycline, Tetracycline, and others; Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in U.S.), Timidazole, or a combination thereof. In one aspect, the therapeutic agent can be a combination of Rifampicin (Rifampin in U.S.) and Minocycline.

Growth factors useful as therapeutic agents include, but are not limited to, transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

Cytokines useful as therapeutic agents include, but are not limited to, cardiotrophin, stromal cell derived factor, macrophage derived chemokine (MDC), melanoma growth stimulatory activity (MGSA), macrophage inflammatory proteins 1 alpha (MIP-1alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, and TNF-β. Immunoglobulins useful in the present disclosure include, but are not limited to, IgG, IgA, IgM, IgD, IgE, and mixtures thereof. Some preferred growth factors include VEGF (vascular endothelial growth factor), NGFs (nerve growth factors), PDGF-AA, PDGF-BB, PDGF-AB, FGFb, FGFa, and BGF.

Other molecules useful as therapeutic agents include but are not limited to growth hormones, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 2, 3, 4, 5 6, 7, 8, 9, 10, 11, 12,13, 15, 16, 17 and 18.

In some embodiments, the therapeutic agent may include active agents susceptible to abuse. Active agents susceptible to abuse can be drugs or salts thereof that have a potential to be abused or which are susceptible to abuse. Substance abuse, sometimes known as drug abuse, is a habitual use of a drug in which the user consumes the substance in amounts, or with methods, which are harmful to the user or others. For example, opioid use disorder is a medical condition that is characterized by the compulsive use of opioids despite adverse consequences from continued use and the development of a withdrawal syndrome when opioid use stops. It involves both an addiction to, and dependence upon, opioids. Opioids can induce physical chemical dependency, behavioral dependency and tolerance. Aside from the physical aspects of chemical dependency and tolerance, in a relatively small number of cases opioids have been associated with iatrogenic (physician-induced) addiction.

Opioid dependence requires long-term treatment and care with the goals of reducing health risks for the consumer, reducing criminal behavior, and improving the long-term physical and psychological condition of the addicted person. Most strategies aim ultimately to reduce drug use and lead to abstinence. No single treatment works for everyone, so several strategies have been developed including therapy and drugs. Detox programs are rarely a good solution, and patients often relapse after going through them. Additionally, the riskiest time for overdose and death for opioid dependent patients occurs within (90) ninety days of discharge from either incarceration or rehabilitation and detoxification.

Opioid replacement therapy (ORT) (also called opioid substitution therapy or opioid maintenance therapy) involves replacing an illegal opioid, such as heroin, with a longer acting but less euphoric opioid; methadone or buprenorphine are typically used and the drug is taken under medical supervision. The driving principle behind ORT is the program's capacity to facilitate a resumption of stability in the user's life, while the patient experiences reduced symptoms of drug withdrawal and less intense drug cravings; a strong euphoric effect is not experienced as a result of the treatment drug. Some patients maintain complete abstinence from opioids while receiving opioid replacement therapy, and others are able to reduce their use. Clonidine or lofexidine can help treat the symptoms of withdrawal.

Active agents susceptible to abuse can include, but are not limited to, those commonly prescribed for relieving pain such as barbiturates and opioids. Opioids are a class of analgesic compounds used medicinally for effective relief of both acute and chronic pain. Opioids bind with the μ G-protein coupled receptor located on the membrane of cells in the brain. Upon binding, opioids act as an agonist, activating the receptor and relieving pain. In addition, opioids are known for alleviating anxiety, inducing mild sedation, and producing a sense of “well-being.”

Active agents susceptible to abuse can include a few drug compounds for pain relief include, but are not limited to, codeine, phenazocine, tilidine, tramadol, meperidine, sufentanil, prodine, methadone, pentazocine, oxycodone, oxymorphone, hydrocodone, hydromorphone, tapentadol, morphine, buprenorphine, and fentanyl. Other drugs that can be misused for non-therapeutic purposes have hallucinogenic properties or otherwise affect the central nervous system, including stimulants such as amphetamines.

Some other drugs that can be the subject of abuse include, but are not limited to, alfentanil; allobarbital; allylprodine; alphaprodine; alprazolam; amfepramone; amphetamine; amphetaminil; amobarbital; anileridine; atomoxetine; apocodeine; barbital; benzodiazepine, benzylmorphine; bezitramide; bromazepam; brotizolam; buprenorphine butobarbital; butorphanol; buspirone: camazepam: carisoprodol, chlorodiazepoxide; clobazam; clonazepam; clonitazene; clorazepate; clotiazepam; cloxazolam; cocaine; codeine, cyclobarbital; cyclorphan; cyprenorphine; delorazepam; desomorphine; dextroamphetamine, dexmethylphenidate, dextromoramide; dextropropoxyphen; dezocine; diampromide; diamorphone; diazepam; dihydrocodeine; dihydromorphine; dimenoxadol; dimepheptanol; dimethylthiambutene; dioxaphetyl butyrate; dipipanone; dronabinol; eptazocine; ephedrine, estazolam; eszopiclone, ethoheptazine; ethylmethylthiambutene; ethyl loflazepate; ethylmorphine; etonitazene; etorphine; fencamfamine; fenethylline; fenproporex; fentanyl, fludiazepam; flunitrazepam; flurazepam; guanfacine; gabapentin; halazepam; haloxazolam; heroin; hydrocodone, hydromorphone, hydroxypethidine; hydroxymethyl morphinane; isomethadone; ketazolam; ketobemidone; levomethadyl acetate; levomethadone; levorphanol; levophenacylmorphane; lofentanil; loprazolam; lorazepam; lormetazepam; lisdexamfetamine; mazindol; medazepam; mefenorex; meprobamate; meptazinol; metazocine; methadone, methylmorphine; methamphetamine; methaqualone; methylphenidate; methylphenobarbital; methyprylon; meperidine, metopon; midazolam; modafinil; morphine, myrophine; nabilone; nalbuphine; nalorphine; narceine; nicomorphine; nimetazepam; nitrazepam; nordazepam; norlevorphanol; normethadone; normorphine; norpipanone; opium; oxazepam; oxazolam; oxycodone, oxymorphone, pernoline; pentazocine, pentobarbital; pethidine; phenadoxone; phenomorphan; phenoperidine; piminodine; pholcodine; phenmetrazine; phenobarbital; phentermine; phenazocine, pinazepam; pipradrol; piritramide; prazepam; pregabalin; prodine, profadol; proheptazine; promedol; propenidine; propoxyphene; pseudoephedrine, remifentanil; secbutabarbital; secobarbital; serdexmethylphenidate; sufentanil, tapentadol, temazepam; tetrazepam; tilidine; tramadol; triazolam; vinylbital; zolpidem, or any combination thereof. The drugs include any pharmacologically active stereoisomeric compounds, as well as derivatives of the base drug such as esters and salts, including any solvates thereof. The active agent susceptible to abuse can be present in the composition in an amount effective for the intended therapeutic purpose. These amounts are well known in the art. All of the active agents embraced by the present disclosure are known per se, as are the doses at which they can be given safely and effectively for the intended therapeutic purpose.

In some embodiments, the drug delivery devices described herein are made by the methods described herein.

### Methods of Making

Disclosed herein are also methods for preparing a drug delivery device including a matrix with two closed ends including a wall and a lumen extending therethrough from a first end to a second end, and an active agent disposed within the lumen of the matrix, the method comprising: forming a matrix by forming a wall on a rod, the wall including a biodegradable polymer and optionally a non-biodegradable polymer; sintering the matrix following forming the wall; injecting an active agent dispersed in a carrier into the lumen of the matrix; and sealing the first end and the second end to form the drug delivery device. Forming the wall can include electrospinning using a solution of the biodegradable polymer and optionally a non-biodegradable polymer. In some embodiments, forming the wall can include electrospinning using a solution of the biodegradable polymer. In some embodiments, forming the wall can include electrospinning using a solution of the biodegradable polymer and a non-biodegradable polymer. In some embodiments, forming the wall can include electrospinning using a solution of the biodegradable polymer and optionally a non-biodegradable polymer and a voltage difference of from 10 kV to 30 kV, for example from 10 kV to 20 kV, or from 15 kV to 25 kV.

In some embodiments, sintering can include heating at a temperature of from 50° C. to 150° C., for example from 90° C. to 110° C. for a period of from 1 minute to 6 hours, for example from 30 minutes to 6 hours. Sub- or supercritical CO₂ exposures may also be used to enable densification of these polymers to avoid higher temperatures and potentially oxidizing temperatures.

In some embodiments, the method can further include washing the matrix following sintering. In some embodiments, the matrix is washed with a saturated sodium bicarbonate solution followed by deionized water. In some embodiments, the porogen is substantially removed from the drug delivery device upon washing with deionized water.

In some embodiments, the method can further include drying the matrix following washing. In some embodiments, drying can be in vacuo at a temperature of from 50° C. to 150° C., for example from 90° C. to 110° C., for a period of from 1 minute to 6 hours, for example from 30 minutes to 6 hours. In some embodiments, injecting can be performed after sintering. In some embodiments, the injecting step can be performed after drying.

In some embodiments, the method can further include culturing a population of cells on the wall of the matrix. In some embodiments, the method can further include culturing a population of cells on the wall of the matrix prior to injection of the active agent into the lumen of the matrix. In some embodiments, the method can further include culturing a population of cells on the wall of the matrix prior to the sealing the first end and second end. In some embodiments, the cells are stem cells, endothelial cells, mesenchymal cells, kidney cells, pancreatic cells, liver cells, or specific cancer cells engineered to produce desirable compounds or microvesicles.

In some aspects, the two ends of the matrix are closed. The ends may be closed by any number of sealing techniques as would be appropriately selected by one of skill in the art. In some embodiments, the two ends are sealed using a high frequency tube sealing technique. In such techniques, a high frequency generates an eddy current in the wall, which heats up at least the polymer layers. When the temperature has reached the melting point of the polymer, clamps are closed and the melted polymer is cooled and formed. In some embodiments, the two ends are sealed using hot-jaw tube sealing, where heated jaws apply heat to the outside of the shape to heat up the inside for sealing. In some embodiments, the two ends may be sealed using ultrasonic tube sealing. In such techniques, the polymer composition of the inner layers is heated and melted by high frequency friction force introduced form an ultrasonic horn. Clamps are then closed around the section intended to be sealed, cooled, and formed to seal the ends. In some embodiments, the two ends are sealed using hot air sealing, wherein the system heats the seal area inside the capsule with hot air and then subsequently presses and chills the ends in a subsequent station.

In some embodiments, forming the wall can include electrospraying using a solution of the biodegradable polymer and a non-biodegradable polymer. In some embodiments, the solution can further include a porogen. In some embodiments, the solution includes the biodegradable polymer and the non-biodegradable polymer.

The solution includes the biodegradable polymer and the non-biodegradable polymer, the biodegradable polymer can be present in a concentration of at least 5% in the solution, (e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%).

The biodegradable polymer can be present in a concentration of 95% or less in the solution, (e.g., 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, or 10% or less).

The biodegradable polymer can be present in a concentration ranging from any of the minimum values described above to any of the maximum values described above. For example, in some embodiments, when the solution includes the biodegradable polymer and the non-biodegradable polymer, the biodegradable polymer can be present in a concentration of from 5% to 95% in the solution, (e.g., from 5% to 10%, from 5% to 20%, from 5% to 30%, from 5% to 40%, from 5% to 50%, from 5% to 60%, from 5% to 70%, from 5% to 80%, from 5% to 90%, from 10% to 20%, from 10% to 30%, from 10% to 40%, from 10% to 50%, from 10% to 60%, from 10% to 70%, from 10% to 80%, from 10% to 90%, from 20% to 30%, from 200/% to 40%, from 20% to 50%, from 20% to 60%, from 20% to 70%, from 20% to 80%, from 20% to 90%, from 30% to 40%, from 30% to 50%, from 30% to 60%, from 30% to 70%, from 30% to 80%, from 30% to 90%, from 40% to 50%, from 40% to 60%, from 40% to 70%, from 40% to 80%, from 40% to 90%, from 50% to 60%, from 50% to 70%, from 50% to 80%, from 50% to 90%, from 60% to 70%, from 60% to 80%, from 60% to 90%, from 70% to 80%, from 70% to 90%, from 80% to 90%,).

The solution includes the biodegradable polymer and the non-biodegradable polymer, the non-biodegradable polymer can be present in a concentration of at least 5% in the solution, (e.g., at least 10%, at least 15%, at least 20%, at least 25%, at least 30%, at least 35%, at least 40%, at least 45%, at least 50%, at least 55%, at least 60%/, at least 65%, at least 70%, at least 75%, at least 80%, at least 85%, or at least 90%).

The non-biodegradable polymer can be present in a concentration of 95% or less in the solution, (e.g., 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, or 10% or less).

The non-biodegradable polymer can be present in a concentration ranging from any of the minimum values described above to any of the maximum values described above. For example, insome embodiments, when the solution includes the biodegradable polymer and the non-biodegradable polymer, the non-biodegradable polymer can be present in a concentration of from 5% to 95% in the solution, (e.g., from 5% to 10%, from 5% to 20%, from 5% to 30%, from 5% to 40%, from 5% to 50%, from 5% to 60%, from 5% to 70%, from 5% to 80%, from 5% to 90%, from 10% to 20%, from 10% to 30%, from 10% to 40%, from 10% to 50%, from 10% to 60%, from 10% to 70%, from 10% to 80%, from 10% to 90%, from 20% to 30%, from 20% to 40%, from 20% to 50%, from 20% to 60%, from 20% to 70%, from 20% to 80%, from 20% to 90%, from 30% to 40%, from 30% to 50%, from 30% to 60%, from 30% to 70%, from 30% to 80%, from 30% to 90%, from 40% to 50%, from 40% to 60%, from 40% to 70%, from 40% to 80%, from 40% to 90%, from 50% to 60%, from 50% to 70%, from 50% to 80%, from 50% to 90%, from 60% to 70%, from 60% to 80%, from 60% to 90%, from 70% to 80%, from 70% to 90%, from 80% to 90%,).

In some embodiments, when present the biodegradable polymer, the non-biodegradable polymer, and porogen are present in a ratio of from 70:10:20 to 88:10:2 such as 70.10:20, 80:10:10, 82:10:8, 85:10:5, or 88:10:2 in the solution, respectively.

In some embodiments, the biodegradable polymer can include a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof. In some embodiments, natural biodegradable materials (collagen, gelatin, etc.) may be partially or completely crosslinked, e.g., by exposure to glutaraldehyde vapor. In some embodiments, the biodegradable polymer include polycaprolactone (PCL).

In some embodiments, the non-biodegradable polymer. In some embodiments, the non-biodegradable polymer can include, but is not limited to, polyethylene terephthalate (PET), polyvinyl chloride (PVC), polyethylene (PE), polysulfone (PSU), polyethersulfone (PES), polypropylene (PP), polystyrene (PS), poly(urethanes), poly(acrylates), poly(ethylene vinyl acetate), nylon, copolymers, or blends thereof. In some embodiments, the non-biodegradable polymer include polyethylene terephthalate (PET).

A “porogen” as used herein refers to any material that can be used to create a porous material, e.g. porous polycaprolactone as described herein. In some embodiments, the porogen can include a water-soluble compound, i.e. such that the porogen is substantially removed from the outer layer upon washing the drug delivery device with water. In some embodiments, the porogen can include a soluble organic salt such as HEPES salt; biocompatible soluble inorganic salts such as NaCl or KCl; or any combination there of. In some embodiments, the porogen can include a compound selected from ([Tris(hydroxymethyl)methylamino]propanesulfonic acid) (TAPS), (2-(Bis(2-hydroxyethyl)amino)acetic acid) (Bicine), (Tris(hydroxymethyl)aminomethane) or, (2-Amino-2-(hydroxymethyl)propane-1,3-diol) (Tris), (N-[Tris(hydroxymethyl)methyl]glycine) (Tricine), (3-[N-Tris(hydroxymethyl)methylamino]-2-hydroxypropanesulfonic acid) (TAPSO), (4-(2-hydroxyethyl)-1-piperazineethanesulfonic acid) (HEPES), (2-[[1,3-dihydroxy-2-(hydroxymethyl)propan-2-yl]amino]ethanesulfonic acid) (TES), (3-(N-morpholino)propanesulfonic acid) (MOPS), (Piperazine-N,N′-bis(2-ethanesulfonic acid)) (PIPES), Dimethylarsenic acid, (2-(N-morpholino)ethanesulfonic acid) (MES), or salts thereof, such as the sodium salts thereof.

In other embodiments, the disclosed drug delivery device may be manufactured by any appropriate method as would be readily understood by those of ordinary skill in the art. In some embodiments, the disclosed drug delivery device may be manufactured by asymmetric membrane formation; a representative example of such methods are provided in Yen, C. et al. “Synthesis and characterization of nanoporous polycaprolactone membranes via thermally- and nonsolvent-induced phase separations for biomedical device application” Journal of Membrane Science 2009, 343:180-88, hereby incorporated herein by reference in its entirety for all purposes. In some embodiments, the disclosed drug delivery device may be manufactured using three-dimensional printing. In some embodiments, the disclosed drug delivery device may be manufactured around methylcellulose which is subsequently removed to form the luminal compartment. In some embodiments, the disclosed drug delivery device may be manufactured by a method described by Envisia Therapeutics in WO 2015/085251, WO 2016/144832, WO 2016/196365, WO 2017/015604, WO 2017/015616, or WO 2017/015675, each of which is hereby incorporated by reference in its entirety for all purposes. In yet other embodiments, the disclosed drug delivery device may be manufactured by methods similar to those used in the manufacturing of hollow fiber membranes, such as phase inversion including non-solvent induced phase inversion (NIPS), (solvent) evaporation-induced phase inversion (EIPS), vapor sorption-induced phase inversion (VIPS), and thermally induced phase inversion (TIPS) In some embodiments, the disclosed drug delivery device may be manufacturing using a method similar to the methods described in US 2015/232506, incorporated herein by reference in its entirety for all purposes. In some embodiments, the pores may instead by formed by laser diffraction of the drug delivery device.

Described herein are also methods of forming a graft for implantation into a subject, the method can include culturing a population of cells from a subject on the wall of the drug delivery device to form a graft for implantation into the subject. In some embodiments, the cells are stem cells, endothelial cells, mesenchymal cells, kidney cells, pancreatic cells, liver cells, or specific cancer cells engineered to produce desirable compounds or microvesicles. In some embodiments, culturing is performed in a bioreactor.

The details of one or more embodiments of the disclosure are set forth in the accompanying drawings and the description below. Other features, objects, and advantages of the disclosure will be apparent from the description and drawings, and from the claims.

### Methods of Use

Described are methods of treating a clinical condition by administration of a drug delivery device described herein. A clinical condition can be a clinical disorder, disease, dysfunction or other condition that can be ameliorated by a therapeutic composition. In some embodiments, the drug delivery devices can be used to deliver therapeutic drugs to a localized region.

In some embodiments, the drug delivery devices can be used for tissue engineering. For example, the drug delivery device can be used for a graft for implantation into a subject. In some embodiments, the drug delivery device can be used for contraceptive treatment. In some embodiments, the drug delivery device can be used for controlling reproductive processes. In some embodiments, the drug delivery device can be used for cancer treatment. In some embodiments, the drug delivery device can be used to treat opioid dependence. In some embodiments, the drug delivery device can be used deliver an active agent susceptible to abuse.

Described herein are methods of controlling reproductive processes in a subject in need thereof, the method including administering to the subject a drug delivery device described herein including an effective amount of an active agent effective to control reproductive processes. In some embodiments the active agent can be a contraceptive.

In some embodiments, described herein are also methods for contraceptive treatment in a subject in need thereof, the method including administering to the subject a drug delivery device described herein including an effective amount of a contraceptive.

Described herein are methods of treating cancer in a subject in need thereof, the method includes administering to the subject a drug delivery device described herein including an effective amount of an anticancer agent.

Described herein are methods for delivering an active agent susceptible to abuse to a subject in need thereof, the method including administering to the subject a drug delivery device described herein including an effective amount of an active agent susceptible to abuse.

In some embodiments, described herein are methods for treating opioid dependence, the method including administering a drug delivery device described herein including an effective amount of an opioid to a subject in need thereof.

### Examples

### Example 1: All-In-One (AIO) Scaffolds For Tissue Engineering and Drug/Biologicals Delivery

Tissue engineering scaffolds have long been used to provide mechanical and chemical support to mammalian cells for purposes of organ regeneration. However, their ability to deliver drugs and or cells within their interior to a site of clinical interest has been limited by the fact that the same space that allows ‘packaging’ of these drugs or cells compromises their mechanical properties. Use of sintering, can create ‘space’ within scaffolds having a variety of polymer compositions into which substantial amounts of either drugs or cells can be easily placed while simultaneously avoiding significant degradation during the period of operation. This creates an “all in one” capability currently absent from the existing range of tissue engineering scaffolds and drug release vehicles. The result can be a polymer capsule that releases drugs via diffusion through the carefully controlled chemical composition, the specific crystalline architecture of the wall, and/or incorporation of pores for active ingredient diffusion. Such a polymer maybe a blend of two or more polymers having dramatically different melting points that cannot be formed via standard extrusion techniques as the lower melting component(s) of one or more of the components could decompose at the temperatures necessary to form a uniform melt having a viscosity low enough to allow for molding.

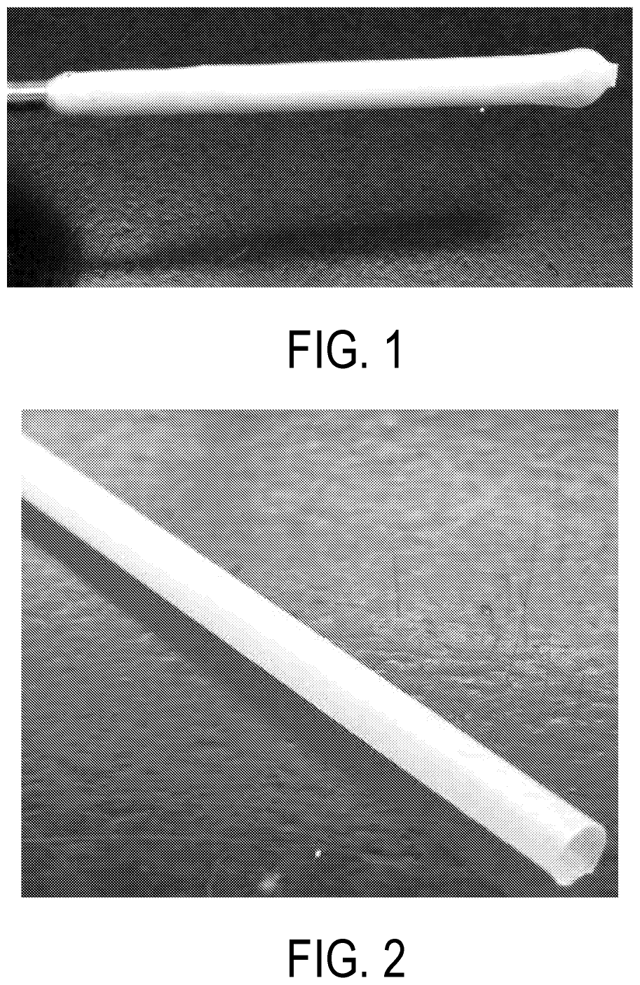

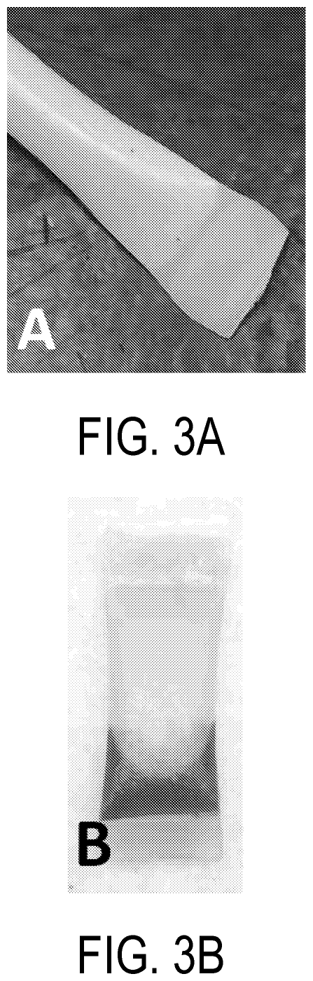

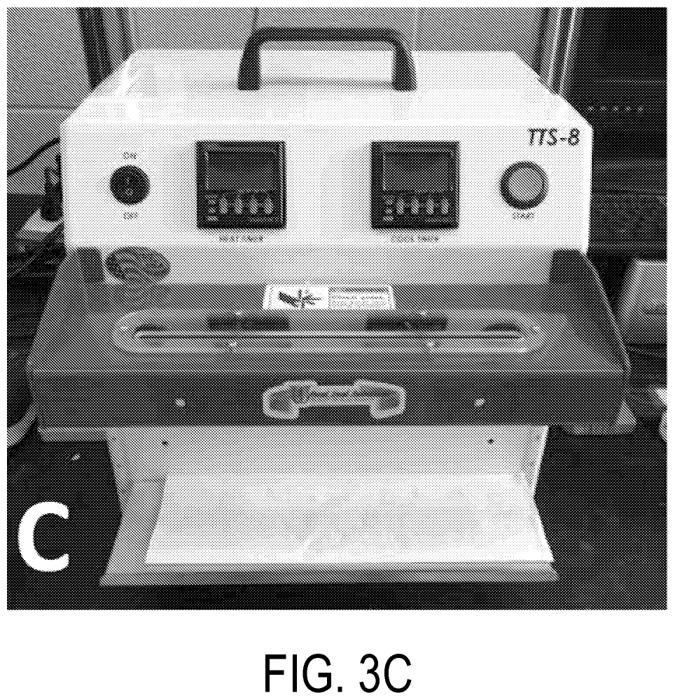

To create a generic advantage that dramatically enhances the long-term drug delivery properties of electrospun scaffolds beyond those currently available via electrospinning alone. A little-known aspect of materials processing is that the shrinkage of high surface area polymers (e.g., in electrospun or electrosprayed form) can be used to provide higher density polymers. In the context of electrospun tissue engineering scaffolds, this phenomenon can be used to create relatively dense material from polymer compositions that would be difficult—if not impossible—to create via extrusion. The relatively dense material then creates a solid barrier through which diffusion of the internally-held drug can occur. The simplest version of this process involves creating a hollow tube via electrospinning (FIG. 1), sintering it to full or nearly-full density (thus eliminating open porosity, see FIG. 2), and then inserting the drug of interest contained with the appropriate solvent. Once this is complete, the ends of the tube can be heat-sealed (FIG. 3) using commonly available, purpose-built equipment designed and manufactured for the purpose of heat-sealing plastics of nearly any chemical composition.

The characteristics of the sintering process itself can be used to provide additional utility. At low temperatures immediately following electrospinning or electrospraying, the polymers should have minimal amounts of crystallinity due to the lack of thermal energy necessary to drive the crystallization process. Less crystallinity should result in faster diffusion through the wall. If sintering can be controlled to allow for full densification but the overall thermal exposure minimized, this can avoid crystallization and simultaneously maximize small molecule diffusion through the wall.

These unique, sintering-structured drug delivery capsules can meet both the structural and functional requirements required by drug delivery vehicles implanted into mammalian tissues. For example, a patient's own cells can be seeded within the walls of the scaffold prior to sealing.

Similarly, the internal chamber could be filled with a pharmaceutical of interest (chemotherapeutic, contraceptives, growth factors, etc) that would then be released through the same diffusional process to affect the surroundings either systemically or locally. In comparison to other forms of drug release which suffer from scar formation and a “walling off” process that limits their effectiveness, AIO implants would be coated in a layer of nanofiber that may or may not be preseeded with stem cells to avoid/minimize the foreign body response. This could be particularly useful as a tissue engineering approach that provides slow, very long-term (years) drug release from a central chamber or chambers.

In FIG. 3 we are examining the diffusive release of a useful indicator, Rose Bengal, through a polycaprolactone (PCL) wall formed by the process of electrospinning followed by sintering to full density. At 973.67 g/mol, Rose Bengal has a molecular size similar to that of many useful pharmaceuticals (deslorelin, 1,282.45 g/mol; histrelin, 1,323.5 g/mol; tamoxifen; 371.51456 g/mol; everolimus, 958.224 g/mol) for which delivery over a period of years can be clinically useful.

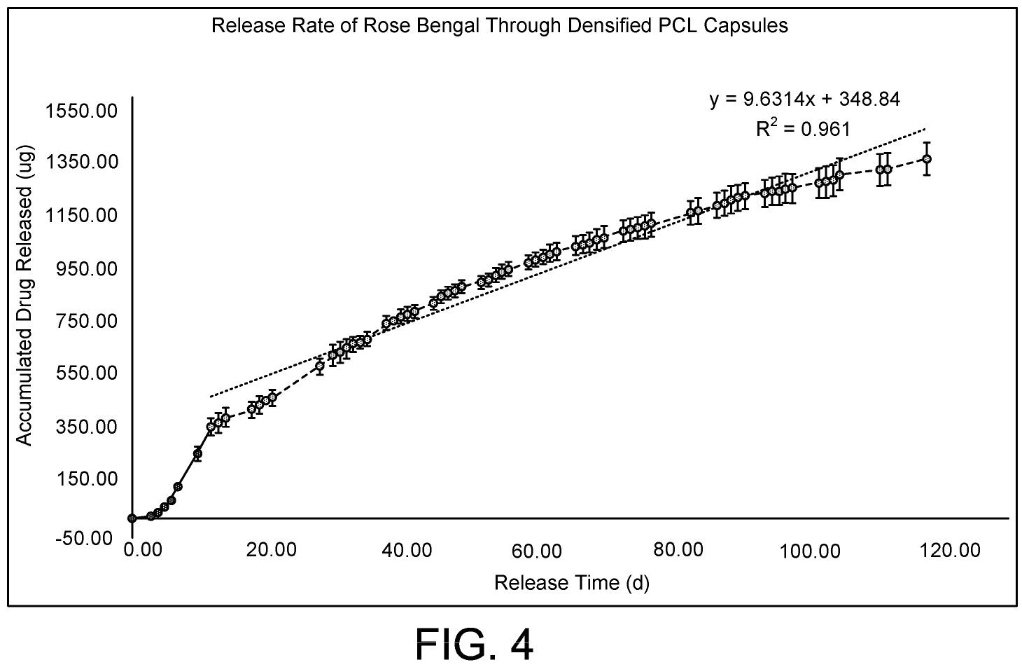

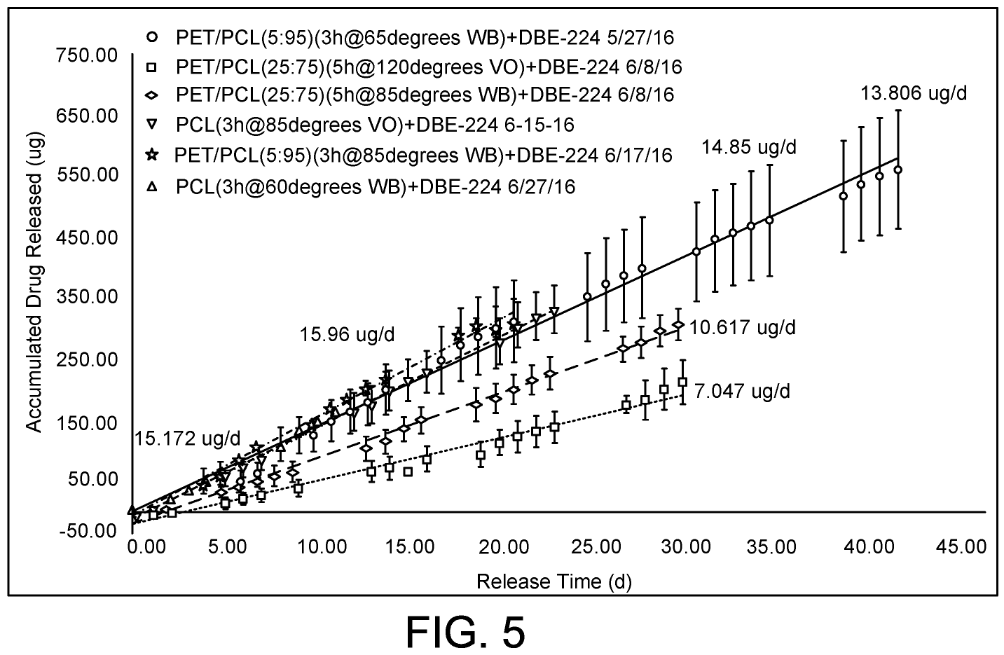

FIG. 4 shows that even after 120 days of exposure to 37° C. phosphate-buffered saline (PBS), 9-10 μg/day of Rose Bengal can continue to diffuse through the PCL. This is more than enough to affect either local or distant biological processes sensitive to the presence of highly biologically active compounds. Similarly, FIG. 5 show the diffusive release of Rose Bengal through a 95% PCL-5% polyethylene terephthalate (PET) wall formed by the process of electrospinning followed by sintering to full density. Here, the inclusion of PET carries with its substantial value as it considered to be a non-biodegradable compound the presence of which will increase the lifetime of the wall during long-term exposure to the hostile biological environment. In this context, the PET can also disrupt the inherent crystallinity of the PCL to enhance diffusional drug release versus PCL without the presence of PET. That amorphous polymers allow greater chemical diffusion than those heavily populated with crystalline domains is well known. This constitutes yet another advantage of electrospinning as a means of creating dense polymer capsules for high rates of drug release over extended periods of time.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A drug delivery device comprising:
a matrix with two closed ends comprising a wall and a lumen extending therethrough from a first end to a second end, and an active agent disposed within the lumen of the matrix;
wherein the wall of the matrix is formed from a biodegradable polymer;
wherein the matrix has a porosity of at least 5%, as determined by mercury porosimetry or apparent density, a density of at least 0.25 g/c, as determined by mercury porosimetry or apparent density, or a combination thereof.

2. The drug delivery device of claim 1, wherein the biodegradable polymer comprises a polyester, polylactic acid (PLA), polyglycolic acid (PGA), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, or blends thereof.

3. (canceled)

4. The drug delivery device of claim 1, wherein the wall of the matrix further comprises a non-biodegradable polymer.

5. The drug delivery device of claim 1, wherein the matrix has a wall thickness of from 25 microns to 1500 microns.

6. The drug delivery device of claim 1, wherein the lumen has a diameter of from 100 μm to 6.0 mm.

7. The drug delivery device of claim 1, wherein the matrix has a length of from 0.1 cm to 20 cm.

8. The drug delivery device of claim 1, wherein at least a portion of the device comprise a coating comprising a population of cells.

9. (canceled)

10. The drug delivery device of claim 1, wherein the wall further comprises a plurality of pores, wherein the pores have an average diameter of from 100 nm to 10 μm.

11. The drug delivery device of claim 1, wherein the active agent is present in an amount of from 1 μg/ml to 100,000 μg/ml.

12. The drug delivery device of claim 1, wherein drug delivery device releases the active agent over a period of at least 3 days, at least 30 days, at least 3 months, at least 6 months when incubated in phosphate-buffered saline at 37° C.

13. A method for preparing a drug delivery device comprising a matrix with two closed ends comprising wall and a lumen extending therethrough from a first end to a second end, and an active agent disposed within the lumen of the matrix, the method comprising:
forming a matrix by forming a wall on a rod, the wall comprising a biodegradable polymer and optionally a non-biodegradable polymer,
wherein the forming the wall comprises electrospinning using a solution of the biodegradable polymer and optionally a non-biodegradable polymer and a voltage difference of from 10 kV to 30 kV;
sintering the matrix following forming the wall;
injecting an active agent dispersed in a carrier into the lumen of the matrix; and
sealing the first end and the second end to form the drug delivery device.

14. The method of claim 13, wherein the solution comprises a porogen.

15. The method of claim 13, wherein the biodegradable polymer comprises a polyester, polylactic acid (PLA), polyglycolic acid (PGA), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof.

16. (canceled)

17. (canceled)

18. The method of claim 13, wherein when the solution comprises the biodegradable polymer and the non-biodegradable polymer, the biodegradable polymer is present in a concentration of from 5% to 95%, in the solution.

19. The method of claim 13, wherein when the solution comprises the biodegradable polymer and the non-biodegradable polymer, the non-biodegradable polymer is present in a concentration of from 5% to 95%, in the solution.

20. The method of claim 13, wherein sintering comprises heating at a temperature of 50° C. to 150° C. for a period of from 1 minute to 6 hours.

21. The method of claim 13, further comprising washing the matrix following sintering.

22. The method of claim 21, further comprising drying the matrix following washing.

23. The method of claim 22, wherein drying is in vacuo at a temperature of from 50° C. to 150° C. for a period of from 1 minute to 6 hours.

24. The method of claim 13, wherein injecting is performed after sintering.

25. The method of claim 24, wherein injecting is performed after drying.

26. The method of claim 13, further comprising culturing a population of cells from a subject on the wall of the matrix prior to injection of the active agent into the lumen of the matrix.

27. (canceled)

28. A drug delivery device made by the method of claim 13.

29. A method of forming a graft for implantation into a subject comprising:
culturing a population of cells from a subject on the wall of the drug delivery device of claim 1 to form a graft for implantation into the subject.

30. (canceled)

31. (canceled)
